(19) United States
(12) Patent Application Publication
(10) Pub. No.: US 2006/0167269 A1
(43) Pub. Date: Jul. 27, 2006

(54) Compounds and methods for the inhibition of compounds cruzi

(76) Inventors: Andrew D Hamilton, Guildford, CT (US); Wesley C Van Voorhis, Seattle, WA (US); Kohei Yokoyama, Seattle, WA (US); Fredericks S Bucker, Seattle, WA (US); Junko Ohkanda, Tokyo (JP); Michael Gelb, Seattle, WA (US); Jeffrey Lockman, Columbus, OH (US)

Correspondence Address: Henry D Coleman;Coleman Sudol Sapone, 714 Colorado Avenue, Bridgeport, CT, 06605-1601, US

(21) Appl. No.: 10/483,096

(22) PCT Filed: Jul. 11, 2002
(86) PCT No.: PCT/US02/22195

§ 371 Date: Sep. 27, 2004

Related U.S. Application Data
(60) Provisional application No. 60/304,711, filed on Jul. 11, 2001.

(51) Int. Cl. C07D 233/61; A61K 31/4172

(52) U.S. Cl. 548/335.5; 514/400

(57) ABSTRACT

The present invention relates to compounds according to the formula (I): Where R^{A} is a C₁-C₁₀ substituted or unsubstituted linear, branch-chained or cyclic alkyl or alkenyl group or a phenyl group according to the formula (II): R^{B} is a C₁-C₁₀ substituted or unsubstituted linear, branch-chained or cyclic alkyl or alkenyl group or a phenyl group of the formula (III): R¹, R², R³, R⁴, R⁵, R⁶, R⁷, R⁸, R⁹ and R¹⁰ are each independently selected from H, C₁-C₁₀ (preferably a C₁-C₄) alkyl or alkenyl group, CF₃, F, Cl, Br, I, CN, NO₂, NH₂, NHR, NRR, COR (acyl group), OR (hydroxyl or ether group), CO₂R (carboxylic acid or ester group), or COSR (thioester group) where R is H or a C₁-C₁₀ (preferably a C₁-C₄) alkyl or alkenyl group, an unsubstituted or substituted aryl (preferably, phenyl) or heterocycle group, or a (IV) group, where R₃ is H, a C₁-C₁₀ (preferably a C₁-C₄) alkyl, alkenyl, ether or a thioether group; and R¹¹ and R¹² are independently selected from H or a C₁-C₃ alkyl or alkenyl group, or a pharmaceutically acceptable salt thereof and methods for treating infections caused by protozoal, fungal and/or bacterial agents such as Trypanosoma cruzi, Mycobacterium spp., Leishmania spp., Cryptococcus spp., Aspergillus spp., Histoplasma spp., Candida spp., especially Candida albicans, Pneumocystis carinii, Trichophyton spp., Microsporum spp., Malassezia spp., Rhizopus spp., Pseudallescheria spp., Blastomyces dermatitidis and Coccidiodes spp., among others.

### FIELD OF THE INVENTION

The present invention relates to compounds and methods for treating infections caused by protozoal, fungal and/or bacterial agents such as Trypanosoma cruzi, Mycobacterium spp., Leishmania spp., Cryptococcus spp., Aspergillus spp., Histoplasma spp.; Blastomyces dermatitidis, Candida spp. especially Candida albicans, Pneumocystis carinii, Trichophyton spp., Microsporum spp., Malassezia spp., Rhizopus spp., Pseudallescheria spp., and Coccidiodes spp., among others.

### BACKGROUND OF THE INVENTION

Chagas Disease and Trypanosoma cruzi

Chagas Disease was discovered in 1909 by Carlos Chagas. It causes the third largest parasitic disease burden in the world and the largest in the Western hemisphere, currently affecting 16-18 million people throughout Central and South America with over 100 million people living in endemic regions and at risk of infection. The disease is caused by the Trypanosoma cruzi parasite, a zooflagellate protozoon similar to that which causes African sleeping sickness. The parasite is transmitted by a variety of vectors, most notably the large, crawling insect Triatoma infestans, which is known in much of South America as the vinchucha. The insects frequently live in the thatched roofs and cracked adobe walls of the houses common to the affected region; their move into populated regions, which has fueled if not caused the epidemic, was precipitated by the destruction of their natural habitat, the forest, by railroads and other development.¹ They feed on both human and animal blood, and inject a small dose of anesthetic into their victims, which allows them to feed unimpeded for up to thirty minutes. The parasite is not transmitted through the bite, but rather through the insect's fecal matter. The vinchucha defecates shortly after eating, leaving its feces close to the bite irritation; when the victim scratches the bite, fecal matter is rubbed into the open sore causing infection.

The life cycle of T. cruzi involves three primary forms of the parasite: amastigote, trypomastigote, and epimastigote.² Structurally, the three varieties are most easily distinguished from one another through the location of their flagella. The trypomastigote has the origin of its flagellum at its posterior tip, the epimastigote near its center, and the amastigote is lacking an external flagellum. The non-infective epimastigote form lives within the gut of the vinchucha. It multiplies rapidly and serves to maintain the parasite level within the insect. In response to nutritional stress it is transported from the midgut to the rectum of the vinchucha, at which time it differentiates into the metacyclic trypomastigote. The trypomastigote, although non-proliferative, is the infectious form of the parasite in both animals and humans. It has been suggested that the urine of the transmitting vector, which can also cause parasite transmission, induces differentiation from epimastigotes into trypomastigotes.³

Once they are transmitted into humans, the trypomastigotes invade a number of cell types, especially the muscle and nerve cells of the heart and gastrointestinal tract, and transform into the amastigote form. This differentiation takes places after a lag period of 20-30 hours and has been shown to be thermosensitive, although the temperature at which transformation occurs varies among parasitic strains.³ The amastigotes, which are formed when the trypomastigotes are released from their phagolysosomal vacuoles, are the form of the parasite which causes the symptoms of Chagas disease: the amastigotes cluster to form cysts which through their repeated reproduction burst the host cells. The amastigotes also differentiate into trypomastigotes, which are the primary active form of the parasite within the blood. It is these trypomastigotes which are taken up by vinchuchas to repeat the parasitic cycle.

Infection by T. cruzi is generally followed by an 8-10 day incubation period and then by the onset of the acute phase of the disease. Only a small percentage of infected individuals experience symptoms of the acute phase, and the phase is generally only fatal for young children and those with weakened immune systems.⁴ Diagnosis of acute Chagas disease is difficult because the symptoms are common to a variety of common diseases: fever, enlargement of lymph nodes, and myocarditis.⁵ The symptoms which are most associated with Chagas disease is Romaña's Sign, swelling of both the upper and lower eyelids on one eye, and chagomas, painful sores which occur at both the bite site and elsewhere on the body. Rassi, et. al. reports that nearly 75% of patients possess one of these two classic symptoms, but others report percentages of below 25%.¹ The acute stage is the point at which currently available drug therapies function, although these treatments are not very effective.

The acute stage generally ends after 1-2 months, and on occasion the disease is spontaneously cured during this phase. The acute phase can be followed by a rapid onset of cardiopathy, a stage known as the sub-acute phase and which quickly leads to death, but is generally followed by a latent period which can last for decades. This latent period, called the indeterminant period by Carlos Chagas, is defined by the presence of parasitic infection but the absence of symptoms. The indeterminant phase is the terminal stage of the disease for up to 40% of infected individuals; the remainder develop chronic Chagas disease.

The chronic phase has two principal symptomatic pathways, those of benign and malignant evolutions.⁴ Benign evolution is the slow onset of cardiac or digestive symptoms, and can persist without catastrophic consequences for decades. It eventually, however, progresses to malignant chronic Chagas disease, which also can evolve directly from the indeterminant stage of the disease. The malignant form of the disease has two principal components⁵: cardiac and digestive Chagas disease. The cardiac symptoms of Chagas disease have their basis in disruptions of the electronic conduction system of the heart. This degeneration of the heart's conduction system, which is caused by lesions stemming from amastigotic cyst formation within the area, can lead to arrhythmia and bradycardia. These disruptions eventually leads to cardiac failure. Enlargement of the heart is also common, and is occasionally observed in other stages of the disease and can be used as a diagnostic tool.

Digestive decay is slightly less common than cardiac symptoms of Chagas disease, but is more dramatic in its outward symptoms. Nerve damage caused by amastigotic cysts in either the colon or the esophagus diminishes peristalsis, the ability of smooth muscle to dilate and contract in order to move food along the digestive tract. This loss of activity causes muscle hypertrophy which leads to a loss of rigidity and a dramatic enlargement of the affected area. Megaesophagus and megacolon can lead to death due to malnutrition, and furthermore megacolon prevents bowel movements which eventually leads to further digestive failure and eventually results in death. Megaesophagus usually precedes colonic and cardiac symptoms. and is, for unknown reasons, more common among males than females.

Sterol Biosynthesis in Trypanosoma cruzi

Sterol biosynthesis is a complex enzymatic pathway which produces membrane lipids for all eukaryotic organisms. Mammals produce cholesterol as their primary sterol, whereas fungi and trypanosomes produce ergosterol, a similar molecule lacking cholesterol's Δ⁵⁽⁶⁾ double bond and containing a methyl group at C24.⁶ Both cholesterol and ergosterol go through the common intermediate sterol lanosterol, which is formed in several steps from acetyl-CoA. The first of the post-lanosterol processing steps is the removal of a methyl group at C14 and the introduction of a C14-C15 double bond. The enzyme which catalyses this former transformation is lanosterol-C14α-demethylase.

C-14α-demethylase has been extensively studied and characterized in fungal systems. It is a cytochrome P-450 enzyme, and consequently is know as P-450_{14DM}. P-450_{14DM} was first isolated from Saccharomyces cerevisiae.⁷ The enzyme was found to catalyze the removal of the 14α methyl carbon (C32) in the presence of molecular oxygen and NADPH.⁸

The removal of the 14α methyl group proceeds through a series of three successive monooxidations. 32-Hydroxylanosterol was confirmed as an intermediate in the reaction pathway, as it was found to both be a substrate for the enzyme and to bind to the enzyme with greater affinity than lanosterol itself.⁹ Similar results were found for the second intermediate: 32-formyllanosterol¹⁰ The mechanism of the final deformylation is not known conclusively, but both a Baeyer-Villiger rearrangement¹¹ and a radical mechanism¹² have been proposed.

14-Methyl sterols cannot function within cell membranes and consequently the inhibition of P-450_{14DM} is an active area of antifungal research. Azole compounds have been shown to form stochiometric complexes with fungal P-450_{14DM}. A paradigm of inhibitor design has been developed where a potent inhibitor would have to contain both a group capable of binding to the heme iron and a group which can interact with the hydrophobic cavity adjacent to the heme.¹³ Likewise, inhibitors must contain a sterically accessible lone pair and a hydrophobic substituent at N-1 position on the azole ring.¹⁴ It has been suggested that other substitution of the azole ring will not be tolerated.

More recently, crystal structures of the P-450_{14DM} from Mycobacterium tuberulosis bound to 4-phenylimidazole and fluconazole were solved.¹⁵ The results offered more precise evidence of the previous predictions of azole inhibitor binding to the enzyme: the imidazole ring binds perpendicularly to the heme and the aromatic region of the inhibitors participate in hydrophobic interactions with surrounding residues.

This research was supported by NIH grants CA67771 and CA52874. Consequently, the United States government has retained certain rights in the invention.

### OBJECTS OF THE INVENTION

It is an object of the invention to provide novel compounds which exhibit antimicrobial activity, including anti-protozoal and anti-fungal activity.

It is an additional object of the present invention to provide pharmaceutical compositions for the treatment of microbial infections, including protozoal infections such as Chagas, and fungus infections, among others.

It is a further object of the invention to provide methods of treating microbial infections, including protozoal infections and fungus infections, especially including those caused by Trypanosoma spp., especially Trypanosoma cruzi, the causative agent of Chagas disease.

It is yet another object of the invention to provide prophylactic methods for preventing infections by microbial agents, including protozoa and fungi in mammalian subjects or reducing the likelihood that a mammalian subject will contract an infection from one or more of these causative agents.

These and/or other objects of the present invention may be readily gleaned from a reading of the details description of the invention which follows.

### BRIEF DESCRIPTION OF THE FIGURES

FIGS. 1-2 represent certain preferred chemical compounds according to the present invention.

FIGS. 3A and B represent mouse data for compound JJ121. FIG. 3A represents Parasite levels and FIG. 3B represents Survival for treated and control mice. Mice were dosed orally at 50 mg/kg twice daily on days 1-10. They were infected with T. cruzi trypomastigotes (Tulahuen) 2×10³ SQ at day 0. Parasitemia was quantified microscopically on a small drop of tail blood at 400×.

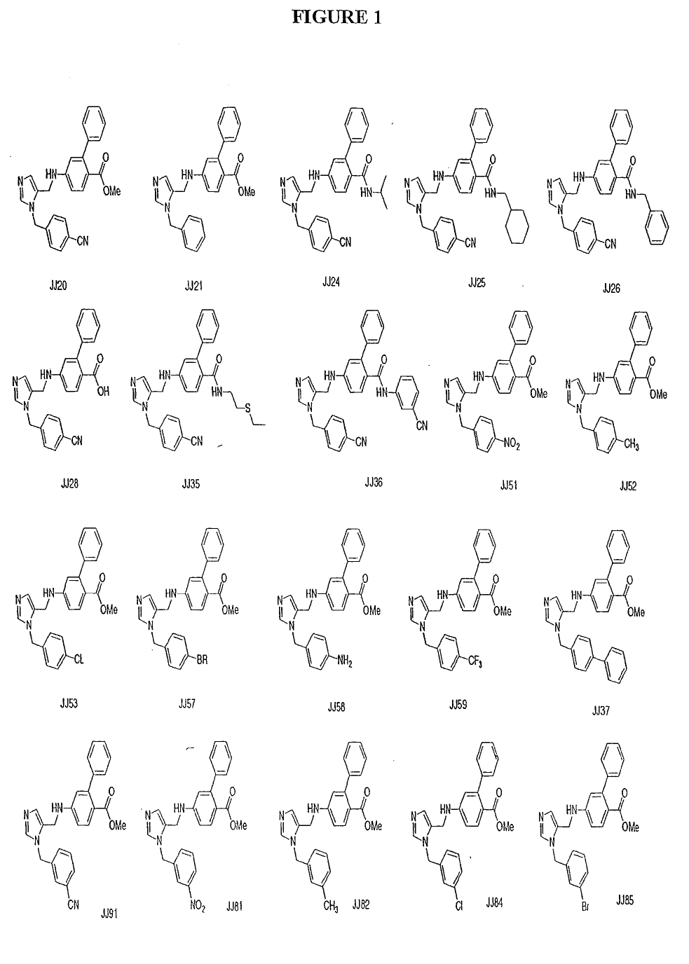

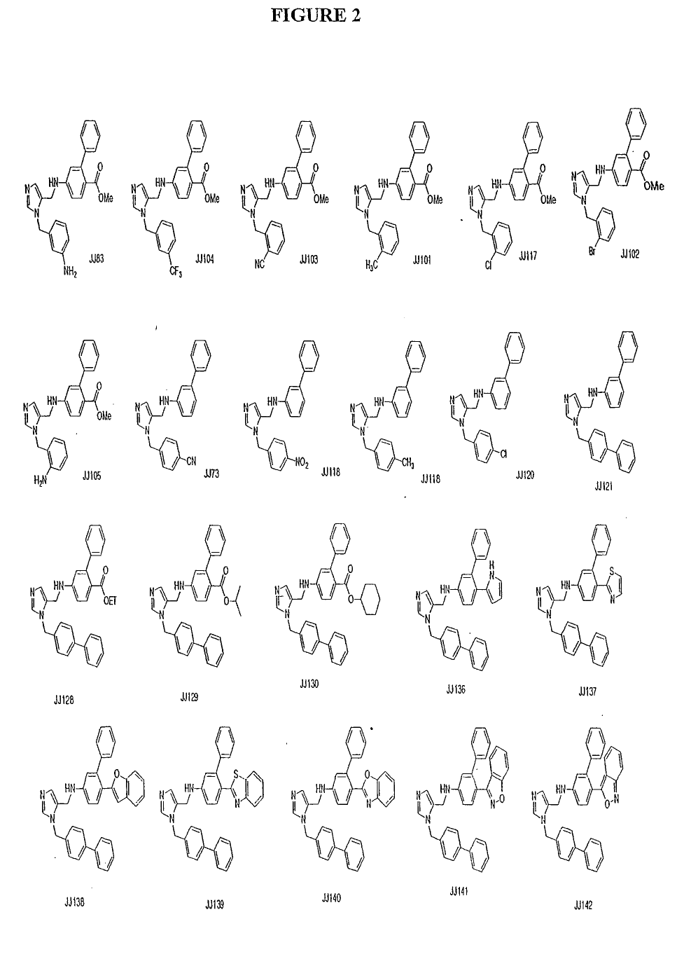

### BRIEF DESCRIPTION OF THE INVENTION

The present invention relates to compounds according to the formula I: 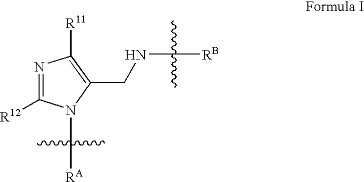
Where R^{A} is a C₁-C₁₀ substituted or unsubstituted linear, branch-chained Or cyclic alkyl or alkenyl group or a phenyl group according to the formula: 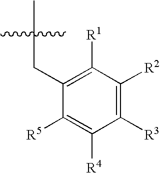
R^{B} is a C₁-C₁₀ substituted or unsubstituted linear, branch-chained or cyclic alkyl or alkenyl group or a phenyl group of the formula: 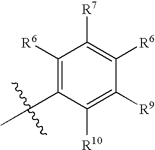
R¹, R², R³, R⁴, R⁵, R⁶, R⁷, R⁸, R⁹ and R¹⁰ are each independently selected from H, C₁-C₁₀ (preferably a C₁-C₄) alkyl or alkenyl group, CF₃, F, Cl, Br, I, CN, NO₂, NH₂, NHR, NRR, COR (acyl group), OR (hydroxyl or ether group), CO₂R (carboxylic acid or ester group), or COSR (thioester group) where R is H or a C₁-C₁₀ (preferably a C₁-C₄) alkyl or alkenyl group, an unsubstituted or substituted aryl (preferably, phenyl) or heterocycle group,
or a 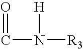
group, where R₃ is H, a C₁-C₁₀ (preferably a C₁-C₄) alkyl, alkenyl, ether or a tioether group; and
R¹¹ and R¹² are independently selected from H or a C₁-C₃ alkyl or alkenyl group, or a pharmaceutically acceptable salt thereof.

Preferably, the heterocycle as set forth above is a furan, pyrrole, imidazole, thiazole, oxazole or isoxazole, all of which may be substituted or unsubstituted, preferably substituted with a phenyl group which may be bonded at one or two carbon atoms of said heterocycle with said phenyl group, said phenyl group being substituted or unsubstituted, preferably unsubstituted. In preferred aspects of the present invention, the heterocycle is bonded with a single unsubstituted phenyl group at two carbon atoms of said heterocycle.

In another aspect of the present invention, pharmaceutical compositions comprise an effective amount of at least one compound as set forth above in pharmaceutical dosage form, optionally in combination with a pharmaceutically acceptable additive, carrier or excipient.

In another aspect of the present invention, methods of inhibiting microbial growth or treating microbial infections and in particular, infections caused by protozoa, especially Trypanosoma cruzi, otherwise known as Chagas, as well as fungal infections and infections having as a causative agent a protozoal, fungal and/or bacterial agent such as Trypanosoma spp., especially T. cruzi, Mycobacterium spp., especially Mycobacterium tuberculosis, Leishmania spp., Cryptococcus spp., Aspergillus spp., Histoplasma capsulatum, Candida spp. especially Candida albicans, Pneumocystis carinii, Trichophyton spp., Microsporum spp., Malassezia spp., Rhizopus spp., Pseudallescheria spp., Blastomyces dermatitidis and Coccidiodes spp., among others, comprise administering to a patient in need of therapy an effective amount of one or more compounds according to the present invention. The present compounds may be used prophylactically to reduce the likelihood that a patient at risk for contracting one or more of the diseases or infections described above by administering an effective amount of one or more compounds according to the present invention to the patient at risk.

In addition to the use of the present compounds to inhibit microbial infections or to treat infections as identified above, the present compounds may also be used in comparison tests such as assays as standard inhibitors of any one or more of the above-isdentified microbes for determining the activities of related anti-microbial compounds as well for determining the susceptibility of a patient's microbial infection to one of the compounds according to the present invention.

### DETAILED DESCRIPTION OF THE INVENTION

The following definitions will be used throughout the specification to describe the present invention.

The term “patient” is used throughout the specification to describe a subject animal, preferably a human, to whom treatment, including prophylactic treatment, with the compositions according to the present invention is provided. For treatment of those infections, conditions or disease states which are specific for a specific animal such as a human patient, the term patient refers to that specific animal.

The term “Trypanosoma” is used throughout the specification to describe the genus of digenetic protozoan flagellates from the family Trypanosomatidae, of which the members have a spindle-shaped body with an undulating membrane on one side, a single anterior flagellum and a kinetoplast. As a rule, these protozoa are parasitic in the blood plasma of vertebrates with only a few being pathogenic. In general members of this genus have an intermediate host, a bloodsucking invertebrate such as a leech, tick or insect. Pathogenic species cause trypanosomiasis in humans and a number of other diseases in domestic animals. The term “Trypanosoma cruzi” refers to the species of Trypanosoma which causes trypanosomiasis and is endemic in Mexico and various countries of Central and South America. The disease which is caused by this causative agent is otherwise known as Chagas disease. In this disease, trypomastigotes are found in the blood and amastigotes occur intracellularly in clusters or colonies in the tissues. Heart muscle fibers and cells of many other organs may be attacked the organisms are not restricted to macrophages as in visceral leishmaniasis. Humans dogs, cats, house rates, armadillos, bats, certain monkeys and opossums are the usual vertebrate hosts. Vectors are members of the family Triatomonia. Also known as Schizotrypanum cruzi, a distinct generic designation widely used in the endemic regions.

The term “Mycobacterium spp.” refers to a genus of aerobic, nonmotile bacteria containing Gram-positive, acid-fast, slender, straight or slightly curbed rods. A number of Mycobacterium associated diseases are associated with immunocompromised patients, especially those with AIDS. Mycobacterium tuberculosis refers to the causative agent of tuberculosis, which may affect any tissue or organ of the body, the most common location of the disease being found in the lungs.

The term “Leishmania spp.” refers to a genus of digenetic, asexual protozoan flagellates of the same family as Trypanosoma that occur as amastigotes in the macrophages of vertebrate hosts and as promastigotes in invertebrate hosts and in cultures. Leishmaniasis refers to infection with a species of Leishmania resulting in a clinically ill-defined group of diseases traditionally divided into four major types: 1) visceral leishmaniasis (kala azar); 2) Old World cutaneous leishmaniasis; 3) New World cutaneous leishmaniasis; 4) mucocutaneous leishmaniasis. Each is clinically and geographically distinct and each has been in recent years been subdivided further into clinical and epidemiological categories. Transmission is by various sandfly species of the genus Phlebotomus or lutzomyia. There are more than 20 species falling within this genus which produce leishmaniasis in mammals, especially humans.

The term “Cryptococcus spp.” refers to a genus of yeastlike fungi that reproduce by budding, certain species of which cause cryptococcosis (a pulmonary, disseminated or meningeal mycosis). Cryptococcus neoformans refers to the species of Cryptococcus which produces cryptococcosis in humans, and other mammalians.

The term “Aspergillus spp.” refers to a genus of fungi in class Ascomycetes that contains many species, a number of them with black, brown or green spores. A few species are pathogenic for humans, other mammals and birds. There are approximately 300 species in this genus. The disease state which is caused by Aspergillus, known as aspergillosis, occurs as a consequence of the presence of Aspergillus in the tissues or on a mucous surface of humans and animals.

The term “Histoplasma capsulatum” refers to a dimorphic fungus species of worldwide distribution that causes histoplasmosis in humans and other mammals. Its ascomycetous state is Ajellomyces capsulatum. “Histoplasmosis” refers to a widely distributed infectious disease caused by Histoplasma capsulatum which occurs frequently in epidemics. Histoplasmosis is often acquired by inhalation of spores of the fungus in soil dust and manifested by a primary pneumonitis similar in clinical features to a mild form of primary tuberculosis. Occasionally, the disease progresses to produce localized lesions in the lung or other clinical manifesations. Histoplasmosis is also known as Darling's disease.

The term “Blastomyces” or “Blastomyces dermatitidis” refers to a dimorphic soil fungus that causes blastomycosis. It grows in mamalian tissues as budding cells and in culture as a white to buff-colored filamentous fungus bearing spherical or ovoid conidia on terminal or lateral short, slender conidophores. In its teleomorph state it is also known as Ajellomyces dermatitidis. “Blastomycosis” refers to a chronic granulomatous and suppurative disease caused by Blastomyces dermatitidis. Blastmomycosis originates as a respiratory infection and disseminates usually with pulmonary, osseous and/or cutaneous involvement predominating. The disease is found in North America, South America and Africa. Gilchrist's disease is also known as blastomycosis.

The term “Coccidiodes spp.” is used to describe a genus of fungi found in the soil of the semi-arid areas of the Southwestern United States and similar areas throughout Central and South America, but has been found elsewhere. The only pathogenic species within the genus is C. immitis, which causes coccidioidomycosis. “Coccidioidomycosis” refers to a variable benign, severe or fatal systemic mycosis due to inhalation of dust particles containing arthroconidia of Coccidioides immitis. In benign forms of the infection, the lesions are limited to the upper respiratory tract and lungs; in a low percentage of cases, the disease disseminates to other visceral organs, bones, joints and skin and subcutaneous tissues. Posadas disease is also known as coccidioidomycosis.

The term “Candida spp.” is used throughout the specification to describe a genus of yeast like fungi commonly found in nature; a few species are isolated from the skin, feces, vagina and pharyngeal tissue, as well as the gastrointestinal tract. Candida albicans is a fungal species which is ordinarily part of a human's normal gastrointestinal flora, but which becomes pathogenic when there is a disturbance in the balance of flora or in the debilitation of the host from other causes. Candida albicans may be associated with septicemia, meningitis and endocarditis. Also known as thrush fungus. Other species of fungi within this genus include Candida krusei, Candida glabrata, Candida tropicalis and Candida parapsilosis. “Candidiasis” is an infection or disease state caused by Candida, especially Candida albicans. Also known as candidosis or moniliasis.

The term “Pneumocystis spp.” and in particular, Pneumocystis carinii” is used to describe the microorganism which causes pneumocystis pneumonia (also referred to as pneumoncystosis) in debilitated patients.

The term “pharmaceutically acceptable salt” is used throughout the specification to describe a salt form of analogs of one or more of the compounds described herein which are presented to increase the solubility of the compound in the gastic juices of the patient's gastrointestinal tract in order to promote dissolution and the bioavailability of the compounds. Pharmaceutically acceptable salts include those derived from pharmaceutically acceptable inorganic or organic bases and acids. Suitable salts include those derived from alkali metals such as potassium and sodium, alkaline earth metals such as calcium, magnesium and ammonium salts, among numerous other acids well known in the pharmaceutical art. Additional salts include acid addition salts of amines such as, for example, HCl salts, carboxylic acid salts (malate, citratre, taurate, oxalate, etc.) and phosphate salts, among numerous others.

The term “pharmaceutically acceptable derivative” is used throughout the specification to describe any pharmaceutically acceptable prodrug form (such as an ester or ether or other prodrug group) which, upon administration to a patient, provides directly or indirectly the present compound or an active metabolite of the present compound.

The term “alkyl” shall mean within its context a fully saturated C₁-C₁₀ hydrocarbon linear, branch-chained or cyclic radical, preferably a C₁-C₄, even more preferably a C₁-C₃ linear, branch-chained or cyclic fully saturated. hydrocarbon radical. The term “alkenyl” is used to describe a hydrocarbon group, similar to an alkyl group which contains at least one double bond.

The term “aryl” shall mean within its context a substituted or unsubstituted monovalent aromatic radical having a single ring (e.g., phenyl) or multiple condensed rings (e.g., naphthyl, anthracene, phenanthrene). Other examples include heterocyclic aromatic ring groups (heteroaryl) having one or more nitrogen, oxygen, or sulfur atoms in the ring, such as imidazolyl, furyl, pyrrolyl, pyridyl, and indolyl. The preferred aryl group is a phenyl or substituted phenyl group.

The term “ether” or “thioether” shall mean a C₁ to C₁₀, (preferably a C₁-C₄) ether or thioether group group, formed from an oxygen or sulfur and an alkyl/alkylene group at a position on phenyl moiety of compounds according to the present invention, or alternatively, may also contain at least one oxygen within the alkyl chain.

The term “heterocycle” shall mean a moiety which is cyclic and contains at least one atom other than a carbon atom, such as a nitrogen, sulfur, oxygen or other atom. Preferably, a heterocycle according to the present invention is a furan, pyrrole, imidazole, thiazole, oxazole or isoxoazole group, which may be substituted or unsubstituted, preferably substituted with a phenyl group which may be bonded at one or two carbon atoms of said heterocycle with said phenyl group (preferably, the phenyl group is bonded to two positions on the heterocycle, thus forming a two membered ring structure), said phenyl group being substituted or unsubstituted, preferably unsubstituted. In preferred aspects of the present invention, the heterocycle is bonded with a single unsubstituted phenyl group at two carbon atoms of said heterocycle.

The term “unsubstituted” shall mean substituted with hydrogen atoms. The term “substituted” shall mean, within the chemical context of the compound defined, a substituent selected from an alkyl, aryl (which also may be heteroaryl), CF₃, halogen, CN, nitro, amine (including monoalkyl and dialkyl amines, acyl, ester, carboxylic acid, thioester, ether, thioether, amide or substituted amide.

The term “inhibitory effective concentration” or “inhibitory effective amount” is used throughout the specification to describe concentrations or amounts of compounds according to the present invention which substantially or significantly inhibit the growth or replication of susceptible microbes, including protozoa and fungi, especially T. cruzi.

The term “therapeutic effective amount” or “therapeutically effective amount” is used throughout the specification to describe concentrations or amounts of compounds according to the present invention which are therapeutically effective in treating various microbial infections in patients, especially including those disease states or conditions having as a causative agent a protozoa or fungus, especially T. cruzi.

The term “preventing effective amount” is used throughout the specification to describe concentrations or amounts of compounds according to the present invention which are prophylactically effective in preventing, reducing the likelihood of contracting or delaying the onset of microbial infections, in particular protozoal or fungal infections or related conditions in patients.

The term “effective amount” shall mean an amount or concentration of a compound or composition according to the present invention which is effective within the context of its administration, which may be inhibitory, prophylactic and/or therapeutic.

Compounds according to the present invention may be synthesized by methods known in the art, or alternatively, by the preferred efficient synthetic methods presented in the present specification. Compounds not specifically presented in the examples section of the present specification may be readily synthesized by analogy with those schemes specifically presented.

Pharmaceutical compositions based upon these novel chemical compounds comprise the above-described compounds in a therapeutically effective amount for treating a microbial infection as described herein, especially a protozoal or fungal infection, especially a T. cruzi infection, optionally in combination with a pharmaceutically acceptable additive, carrier or excipient. One of ordinary skill in the art will recognize that a therapeutically effective amount will vary with the infection or condition to be treated, its severity, the treatment regimen to be employed, the pharmacokinetics of the agent used, as well as the patient (animal or human) treated.

In the pharmaceutical aspect according to the present invention, the compound according to the present invention is formulated preferably in admixture with a pharmaceutically acceptable carrier. In general, it is preferable to administer the pharmaceutical composition in orally-administrable form, but certain formulations may be administered via a parenteral, intravenous intramuscular, transdermal, buccal, subcutaneous, suppository or other route. Intravenous and intramuscular formulations are preferably administered in sterile saline. Of course, one of ordinary skill in the art may modify the formulations within the teachings of the specification to provide numerous formulations for a particular route of administration without rendering the compositions of the present invention unstable or compromising their therapeutic activity. In particular, the modification of the present compounds to render them more soluble in water or other vehicle, for example, may be easily accomplished by minor modifications (salt formulation, esterification, etc.) which are well within the ordinary skill in the art. It is also well within the routineer's skill to modify the route of administration and dosage regimen of a particular compound in order to manage the pharmacokinetics of the present compounds for maximum beneficial effect in patients.

In certain pharmaceutical dosage forms, the pro-drug form of the compounds, especially including acylated (acetylated or other) and ether (alkyl and related) derivatives, phosphate esters and various salt forms of the present compounds, are preferred. One of ordinary skill in the art will recognize how to readily modify the present compounds to pro-drug forms to facilitate delivery of active compounds to a targeted site within the host organism or patient. The routineer also will take advantage of favorable pharmacokinetic parameters of the pro-drug forms, where applicable, in delivering the present compounds to a targeted site within the host organism or patient to maximize the intended effect of the compound.

The amount of compound included within therapeutically active formulations according to the present invention is an effective amount for treating the infection or condition, in preferred embodiments, a protozoal or fungal infection, especially a T. cruzi infection. In general, a therapeutically effective amount of the present compound in pharmaceutical dosage form usually ranges from about 0.05 mg/kg to about 100 mg/kg per day or more, more preferably, slightly less than about 1 mg/kg. to about 25 mg/kg per day of the patient or considerably more, depending upon the compound used, the condition or infection treated and the route of administration. In the case of T. cruzi infections, the active compound is preferably administered in amounts ranging from about 0.5 mg/kg to about 25 mg/kg per day of the patient, depending upon the pharmacokinetics of the agent in the patient. This dosage range generally produces effective blood level concentrations of active compound which may range from about 0.05 to about 100 micrograms/cc of blood in the patient. For purposes of the present invention, a prophylactically or preventive effective amount of the compositions according to the present invention falls within the same concentration range as set forth above for therapeutically effective amount and is usually the same as a therapeutically effective amount.

Administration of the active compound may range from continuous (intravenous drip) to several oral administrations per day (for example, Q.I.D.) and may include oral, topical, parenteral, intramuscular, intravenous, sub-cutaneous, transdermal (which may include a penetration enhancement agent), buccal and suppository administration, among other routes of administration. Enteric coated oral tablets may also be used to enhance bioavailability of the compounds from an oral route of administration. The most effective dosage form will depend upon the pharmacokinetics of the particular agent chosen as well as the severity of disease in the patient. Oral dosage forms are particularly preferred, because of ease of admnistration and prospective favorable patient compliance.

To prepare the pharmaceutical compositions according to the present invention, a therapeutically effective amount of one or more of the compounds according to the present invention is preferably intimately admixed with a pharmaceutically acceptable carrier according to conventional pharmaceutical compounding techniques to produce a dose. A carrier may take a wide variety of forms depending on the form of preparation desired for administration, e.g., oral or parenteral. In preparing pharmaceutical compositions in oral dosage form, any of the usual pharmaceutical media may be used. Thus, for liquid oral preparations such as suspensions, elixirs and solutions, suitable carriers and additives including water, glycols, oils, alcohols, flavouring agents, preservatives, colouring agents and the like may be used. For solid oral preparations such as powders, tablets, capsules, and for solid preparations such as suppositories, suitable carriers and additives including starches, sugar carriers, such as dextrose, mannitol, lactose and related carriers, diluents, granulating agents, lubricants, binders, disintegrating agents and the like may be used. If desired, the tablets or capsules may be enteric-coated or sustained release by standard techniques. The use of these dosage forms may significantly the bioavailability of the compounds in the patient.

For parenteral formulations, the carrier will usually comprise sterile water or aqueous sodium chloride solution, though other ingredients, including those which aid dispersion, also may be included. Of course, where sterile water is to be used and maintained as sterile, the compositions and carriers must also be sterilized. Injectable suspensions may also be prepared, in which case appropriate liquid carriers, suspending agents and the like may be employed.

Liposomal suspensions (including liposomes targeted to microbial antigens) may also be prepared by conventional methods to produce pharmaceutically acceptable carriers. This may be appropriate for the delivery of free compounds or pro-drug forms of the compounds according to the present invention.

In particularly preferred embodiments according to the present invention, the compounds and compositions are used to treat, prevent or delay the onset of microbial infections, especially protozoal or fungal infections and in particular, T. cruzi infections of mammals, especially humans. In its preferred embodiments, the compounds are used to treat infections caused by protozoal, fungal and/or other microbial agents such as Trypanosoma cruzi, Mycobacterium spp., Leishmania spp., Cryptococcus spp., Aspergillus spp., Histoplasma spp., Trichophyton spp., Microsporum spp., Malassezia spp., Rhizopus spp., Pseudallescheria spp., Blastomyces dermatitidis and Coccidiodes spp., among others, in humans. Preferably, to treat, prevent or delay the onset of one or more of these infections, the compositions will be administered in oral dosage formn in amounts ranging from about 250 micrograms up to about 500 mg or more at least once a day, preferably, up to four times a day. The present compounds are preferably administered orally, but may be administered parenterally, topically or in suppository form.

The compounds according to the present invention, because of their low toxicity to host cells, may advantageously be employed prophylactically to prevent a microbial infection or to prevent the occurrence of clinical symptoms associated with the microbial infection. Thus, the present invention also encompasses methods for the prophylactic treatment of microbial infections, and in particular protozoal, fungal and/or bacterial agents such as Trypanosoma cruzi, Mycobacterium spp., Leishmania spp., Cryptococcus spp., Aspergillus spp., Histoplasma spp., Trichophyton spp., Microsporum spp., Malassezia spp., Rhizopus spp., Pseudallescheria spp., Blastomyces dermatitidis and Coccidiodes spp., among others, especially T. cruzi infections. In this aspect according to the present invention, the present compositions are used to prevent reduce the likelihood of or delay the onset of a microbial infection, especially a protozoal or fungal infection or a related disease or condition such as Chagas disease, tuberculosis, leishmaniasis, cryptococcosis, aspergillosis, histoplasmosis, dermatophytosis (caused by Trichophyton spp., Microsporum spp. and Malassezia spp.), mucormycosis, (caused by Rhizopus spp.), Onochomycosis (caused by anyone of several fungi), invasive infections in immunocompromised (caused by Pseudallescheria spp.), blastomycosis and coccidioidomycosis, among others. This prophylactic method comprises administering to a patient in need of such treatment or who is at risk for the development of one or more of a microbial infection, including a protozoal and/or a fungal infection as described herein, or a disease state such as Chagas disease, tuberculosis, leishmaniasis, cryptococcosis, aspergillosis, histoplasmosis, blastomycosis and coccidioidomycosis, among others an amount of a compound according to the present invention effective for alleviating, preventing or delaying the onset of the infection. In the prophylactic treatment according to the present invention, it is preferred that the compound utilized should be as low in toxicity and preferably non-toxic to the patient. It is particularly preferred in this aspect of the present invention that the compound which is used should be maximally effective against the infection and should exhibit a minimum of toxicity to the patient. In the case of compounds of the present invention for the prophylactic treatment of protozoal and/or fungal infections, these compounds may be administered within the same dosage range for therapeutic treatment (i.e., about 250 micrograms up to about 500 mg. or more from one to four times per day for an oral dosage form) as a prophylactic agent to prevent the proliferation of the infection or alternatively, to prolong the onset of or reduce the likelihood of a patient contracting an infection which manifests itself in clinical symptoms.

In addition, compounds according to the present invention may be administered alone or in combination with other agents, including other compounds of the present invention. Certain compounds according to the present invention may be effective for enhancing the biological activity of certain agents according to the present invention by reducing the metabolism, catabolism or inactivation of other compounds and as such, are co-administered for this intended effect.

As indicated, compounds according to the present invention may be administered alone or in combination with other agents, especially including other compounds of the present invention or compounds which are otherwise disclosed as being useful for the treatment of Chagas disease, tuberculosis, leishnianiasis, cryptococcosis, aspergillosis, histoplasmosis, blastomycosis and coccidioidomycosis, among others, including those presently used to treat one or more of these disease states.

Compounds used in the art may be used in combination with the present compounds for their additive activity or treatment profile against Chagas disease, tuberculosis, leishmaniasis, cryptococcosis, aspergillosis, histoplasmosis, blastomycosis and coccidioidomycosis, among others and in certain instances, for their synergistic effects in combination with compounds of the present invention. Preferred secondary or additional compounds for use with the present compounds are those which do not inhibit the causative agents of Chagas disease, tuberculosis, leishmaniasis, cryptococcosis, aspergillosis, histoplasmosis, blastomycosis and coccidioidomycosis, among others by the same mechanism as those of the present invention. Certain compounds according to the present invention may be effective for enhancing the biological activity of certain agents according to the present invention by reducing the metabolism or inactivation of other compounds and as such, are co-administered for this intended effect.

In a particularly preferred pharmaceutical composition and method aspect of the present invention for treating T. cruzi, the causative agent of Chagas disease, an inhibitory effective amount of the present compound is administered to a patient suffering from such an infection to treat the infection and alleviate the symptoms of such infection.

The present invention is now described, purely by way of illustration, in the following examples. It will be understood by one of ordinary skill in the art that these examples are in no way limiting and that variations of detail can be made without departing from the spirit and scope of the present invention.

### EXAMPLES

Synthesis of JJ-Compounds

Representative Procedures:

Imidazolecarboxaldehyde (X-imid-CHO)

1-Triphenylmethyl-5-imidazolecarboxaldehyde (0.5 g, 1.5 mmol) and 4-nitrobenzyl bromide (0.33 g, 1.5 mmol) were added to 10 mL of acetonitrile and stirred overnight at 60° C. The solvent was removed by evaporation and the resulting solid triturated with 15 mL of acetone. The solid was removed by filtration and extracted with dichloromethane (100 mL) and saturated aqueous sodium bicarbonate (60 mL). The aqueous layer was washed with dichloromethane (2×50 mL), dried with sodium sulfate, and concentrated under vacuum. The resulting solid was used without purification (265 mg, 76%). Note that only the para-substituted aldehydes were pure enough for characterization.

Reductive Amination (X-imid-BP-COOMe)

1-(4-nitrobenzyl)-5-imidazolecarboxaldehyde (200 mg, 0.86 mmol) and N-[4-Amino-2-phenylbenzoyl]-methionine methyl ester hydrochloride (254 mg, 0.85 mmol) were added to 14 mL methanol and stirred at rt. for 30 min. under nitrogen in the presence of molecular sieves. Acetic acid (0.5 mL) was added and the solution stirred for 5 min., after which sodium cyanoborohydride (108 mg, 1.74 mmol) was added in portions. The solution was stirred overnight at rt. under nitrogen, after which it was extracted from dichloromethane (130 mL) and saturated aqueous sodium bicarbonate (70 mL). The organic layer was washed with dichloromethane (2×70 mL), dried with sodium sulfate, and concentrated under vacuum. The resulting yellow oil was purified by column chromatography (100:40:8 CHCl₃:acetone:EtOH as eluent) and concentrated to yield a yellow amorphous solid (307 mg, 84%).

Esters (JJ128-JJ129)

Thionyl chloride (734 mg, 6.17 mmol, 3 equiv.) was added dropwise to 2-Phenyl-4-nitrobenzoic acid (500 mg, 2.06 mmol) in ethanol (15 mL) at 0° C. The solution was then heated at reflux overnight and the solvent removed by evaporation. The product was isolated as a yellow oil and used without further purification (533 mg, 95%).

Cyclohexyl Ester (JJ130)

Triethylamine (0.73 g, 7.2 mmol) and cyclohexanol (0.79 g, 7.9 mmol, 1.1 equiv) were added dropwise to 2-Phenyl-4-nitrobenzoic acid (1.75 g, 7.2 mmol), EDCI (1.45 g, 7.6 mmol, 1.05 equiv), and HOBT (0.97 g, 7.2 mmol) in dichloromethane (60 mL) at 0° C. The solution was stirred at room temperature overnight under nitrogen and dichlormethane (150 mL) and 10% HCl was added. The organic layer was separated and washed with saturated aqueous sodium bicarbonate (100 mL) and brine (100 mL) and dried with sodium sulfate. The solvent was removed to yield a yellow oil (2.24 g, 96%).

Reductions

2-Phenyl-4-nitrobenzoic acid methyl ester (2.0 g, 7.8 mmol) and stannous chloride (8.8 g, 39 mmol) were dissolved in 75 mL of ethyl acetate. The solution was refluxed under nitrogen for 2.5 h. Upon cooling, 150 mL of saturated sodium bicarbonate was added. The organic layer was removed and the aqueous layer washed with two 100 mL portions of ethyl acetate. The combined organic layers were dried with sodium sulfate, washed with brine, and the solvent evaporated. A white solid was obtained (1.53 g, 86%).

Heteroterphenyls (JJ136-142)

2-bromo-4-nitroaniline

Bromine (12.4 g, 77.6 mmol) in 50 mL of glacial acetic acid was added dropwise over 3 h. to 4-nitroaniline (10.7 g, 77.6 mmol) in 100 mL of glacial acetic acid. The solution was stirred 30 minutes at room temperature, after which it was poured into 200 mL of ice cold water. The resulting yellow solid was filtered and dissolved in ethyl ether (300 mL). The residual acetic acid was removed and the organic layer washed with saturated aqueous sodium bicarbonate (100 mL) and brine (100 mL) and dried with sodium sulfate. After concentration the yellow solid was purified by recrystalization from methanol to yield a yellow solid (11.71 g, 67%).

2-phenyl-4-nitroaniline

2-Bromo-4-nitroaniline (11.71 g, 53.7 mmol) and phenylboronic acid (6.88 g, 56.4 mmol, 1.05 equiv.) were dissolved in 115 mL of acetone. Potassium carbonate (22.3 g, 161 mmol, 3 equiv.) in 140 mL of water and palladium acetate (0.60 g, 2.7 mmol, 0.05 equiv.) were added. The solution was refluxed overnight under nitrogen. Diethyl ether (500 mL) and 1M HCl (200 mL) were added and both layers were filtered through celite. The layers were separated and the aqueous layer was washed with diethyl ether (2×150 mL). The combined organic layers were dried with sodium sulfate and concentrated to yield an orange solid. The solid was recrystallized from ethyl acetate to give a brick-orange solid (7.56 g, 60%).

2-Phenyl-4-nitrobromobenzene

Sodium nitrite (2.66 g, 38.5 mmol, 1.1 equiv.) was added in portions to 21 mL of concetrated sulfuric acid at room temperature. The suspension was cooled to 10° C. and acetic acid (22 mL) was added dropwise. The mixture was stirred for 20 min. at 10° C. and 2-phenyl-4-nitroaniline (7.56 g, 35 mmol) was added in portions over 30 minutes. The solution was stirred for 2 h. at 10° C. and water (15 mL) was added to clear the suspension. The solution was stirred for 1 h. at room temperature and cupric bromide (13.07 g, 56 mmol, 1.6 equiv.) in 27 mL of 2M HCl was added slowly. The resulting black sludge was stirred for 20 min. at room temperature and 1 h. at 60° C. The solution was added to diethyl ether (200 mL) and washed with water (3×100 mL) and brine (150 mL). The organic layer was dried with sodium sulfate and concentrated to give an orange solid which was purified by recrystallization from methanol to yield a red solid (6.94 g, 71%).

3-(5-Nitro-biphenyl-2-yl)-benzo[o]isoxazole

2-Phenyl-4-nitrobromobenzene (556 mg, 2.0 mmol), Pd(PPh₃)₄ (116 mg, 0.1 mmol), and potassium acetate (294 mg, 3.0 mmol, 1.5 equiv.) were flushed with nitrogen for 5 min. after which N,N-dimethylacetamide (5 mL) was added. The solution was flushed with nitrogen for an additional 5 min. and 1,2-benzisoxazole (0.24 mL, 286 mg, 2.4 mmol, 1.2 equiv.) was added. The solution was stirred at 160° C. overnight. The solution was added to diethyl ether (150 mL) and washed with water (3×100 mL). The organic layer was dried with sodium sulfate and concentrated to yield a black oily solid which was purified by column chromotography (4:1 hexane:ethyl acetate as eluent). A yellow oil was obtained (206 mg, 19%).

Spectral Data

p-NO₂-imid-CHO

¹NMR (CDCl₃): δ 9.73 (s, 1 H, CHO), 8.17 (d, 2 H, ortho to NO₂), 7.89 (s, 1 H, NCHN), 7.83 (s, 1 H, NCHCCHO), 7.31 (d, 2 H, meta to NO₂), 5.63 (s, 2 H, NCH₂Ar).

p-CH₃-imid-CHO

white solid (247 mg, 82%).

¹NMR (CDCl₃): 9.74 (s, 1 H, CHO), 7.81 (s, 1 H, NCHN), 7.72(s, 1 H, NCHCCHO), 7.11-7.16 (m, 4 H, aryl), 5.47 (s, 2 H, NCH2Ar), 2.33 (s, 3 H, CH3).

p-Cl-imid-CHO

yellow solid, 50%.

¹NMR (CDCl₃): 9.73 (s, 1 H, CHO), 7.83 (s, 1 H, NCHN), 7.79 (s, 1 H, NCHCCHO), 7.30 (d, 2 H, ortho to Cl), 7.15 (d, 2 H, meta to Cl), 5.49 (s, 2 H, NCH₂Ar).

p-Br-imid-CHO

yellow oil, 106 mg.

¹H NMR (CDCl₃): 9.67 (s, 1 H, CHO), 7.41 (d, 2 H, ortbo to Br), 7.03 (d, 2 H, meta to Br), 5.45 (s, 2 H, NCH₂Ar), imidazole hydrogens cannot be identified.

p-OMe-imid-CHO

Dark red oil, 47%.

¹NMR (CDCl₃): 9.75 (s, 1 H, CHO), 7.80 (s, 1 H, NCHN), 7.71 (s, 1 H, NCHCCHO), 7.19 (d, 2 H, meta to OMe), 6.86 (d, 2 H, ortho to OMe), 5.44 (s, 2 H, NCH₂Ar), 3.79 (OCH₃).

p-Ph-imid-CHO

¹H NMR (CDCl₃): δ 9.79 (s, 1 H, CHO), 7.85 (s, 1 H, NCHN), 7.75 (s, 1 H, NCHCCHO), 7.54-7.57 (m, 5 H, aryl), 7.46 (t, 2 H, J=7 Hz, aryl), 7.29 (d, 3 H, J=7 Hz, aryl), 5.57 (s, 2 H, CH₂Ar).

VI-10 (2-phenyl-4-nitrobromobenzene)

8.20 (d, J=2 H, 1 H, ortho to NO₂), 8.06 (dd, J=2 Hz and 9 Hz, 1 H, ortho to NO₂), 7.86 (d, J=9 Hz, 1 H, ortho to Br), 7.41-7.49 (m, 5 H, Ar)

VI-42 (NO₂-BP-COOEt)

8.22-8.25 (m, 2 H, ortho to NO₂), 7.92 (bd, J=9 Hz, 1 H, ortho to COOEt), 7.32-7.44 (m, 5 H, Ar), 4.12 (q, J=7 Hz, 2 H, CH₂CH₃), 0.99 (t, J=7 Hz, 3 H, CH₂CH₃)

V-44 (NO₂-BP-COOi-Pr)

8.22-8.26 (m, 2 H, ortho to NO₂), 7.96 (dd, J=14 Hz and 56 Hz, 1 H, ortho to COOPr), 7.32-7.44 (m, 5 Hz, Ar), 4.02 (p, J=6 Hz, 1 H, ⁱPr), 1.20 (d, J=6 Hz, 6 H, ⁱPr)

V-62 (NH₂-BP-COOEt)

7.80 (d, J=9 Hz, 1 H, ortho to COOEt), 7.28-7.37 (m, observed 4 H, expected 5, Ar), 6.65 (dd, J=9 Hz and 2 Hz, 1 H, ortho to NH₂), 6.56 (d, J=2 Hz, 1 H, ortho to NH₂), 4.03 (q, J=7 Hz, 2 H, CH₂CH₃), 0.97 (t, J=7 Hz, 3 H, CH₂CH₃)

V-64 (NH₂-BP-COOi-Pr)

7.82 (dd, J=8 Hz and 56 Hz, 1 H, ortho to COOPr), 7.30-7.39 (m, 5 J, Ar), 6.65 (dd, J=8 Hz and 3 Hz, 1 H, ortho to NH₂), 6.54 (d, J=3 Hz, 1 H, ortho to NH₂), 4.92 (p, J=6 Hz, 0.5 H, ⁱPr), 0.99 (d, J=6 Hz, 3 H, ⁱPr). ⁱPr appears as half what it should be.

VI-40 (NO₂-JJ137)

8.18-8.21 (m, 1 H), 8.15 (m, 1 H), 7.45-7.47 (m, 3 H), 7.33-7.34 (m, 2 H), 6.64 (m, 1 H), 6.53 (m, 1 H), 6.20 (m 1 H)

VI-48 (NH₂-JJ136)

8.56 (s, 1 H), 7.52 (s, 1 H), 7.25-7.34 (m, 4 H), 7.19-7.22 (m, 2 H), 6.70-6.74 (m, 2 H)

VI-58 (NH₂-JJ137)

7.32-7.39 (m, 5 H)

6.71 (dd, J=3 Hz and 8 Hz, 1 H, ortho to NH₂), 6.64 (d, J=3 Hz, 1 H, ortho to NH₂), 6.51 (m, 1 H), 6.15-6.17 (m, 1 H), 6.12-6.14 (m, 1 H)

VI-72 (NH₂-JJ138)

7.81 (d, J=8 Hz, 1 H), 7.35-7.37 (m, 5 H), 7.28-7.31 (m, 4 H), 7.07-7.17 (m, 2 H), 6.86 (dd, J=3 Hz and 8 Hz, 1 H), 6.72 (d, J=3 Hz, 1 H), 5.73 (s, 1 H)

VI-74 (NH₂-JJ139)

7.99 (t, J=9 Hz, 2 H), 7.65 (d, J=8 Hz, 1 H), 7.32-7.42 (m, 5 H), 6.78 (dd, J=2 Hz and 8 Hz, 1 H), 6.65 (d, J=2 Hz, 1 H)

VI-96 (NO₂-JJ141)

8.38 (d, J=3 Hz, 1 H0, 8.20 (dd, J=3 Hz and 9 Hz, 1 H), 7.58-7.62 (m, 3 H), 7.41-7.48 (m, 3 H), 7.35-7.37 (m, 1 H), 7.17 (t, J=8 Hz, 1 H), 7.07 (d, J=9 Hz, 1 H), 6.84 (d, J=9 Hz, 1 H)

VI-98 (NO₂-JJ142)

8.33 (d, J=2 Hz, 1 H, ortho to NO₂), 8.25 (dd, J=2 Hz and 9 Hz, 1 H, ortho to NO₂), 7.57 (t, J=8 Hz, 1 H), 7.45-7.51 (m, 1 H), 7.26-7.32 (m, 4 H), 7.13-7.19 (m, 1 H), 7.05 (d, J=8 Hz, 1 H), 6.58 (d, J=9 Hz, 1 H, ortho to anthranil), 6.41 (t, J=8 Hz, 1 H)

VI-108 (NH₂-JJ140)

7.42-7.51 (m, 4 H), 7.31 (t, J=8 Hz, 3 H), 6.98 (d, J=9 Hz, 1 H), 6.88 (t, J=8 Hz, 1 H), 6.79 (d, J-3 Hz, 1 H), 6.72 (dd, J=3 Hz and 9 Hz, 1 Hz), 6.57 (d, J=9 Hz, 1 H)

(JJ-20).

To a solution of 1-(p-cyano)benzyl-5-imidazolecarboxyaldehyde (52 mg, 0.25 mmol) and 4-amino-2-phenyl-benzoic acicd methyl ester (57 mg, 0.25 mmol) in CH₂Cl₂ (2 mL) was added TiCl₄ (25 mg, 0.13 mmol) at 0° C. After stirring 15 mg, a solution of NaBCNH₃ (16 mg, 0.25 mmol) in MeOH (2 mL) was added. The mixture was stirred at r.t. for 2 h, and the product was extracted with CH₂Cl₂ (50 mL) from sat NaHCO₃ (20 mL). The organic layer was washed with brine and dried (MgSO₄). The crude product was purified by SiO₂ column chromatography with CHCl₃:acetone:EtOH=100:20:4 to afford the product as a white solid (35 mg, 51%).

(JJ-21).

To a solution of 1-benzyl-5-imidazolecarboxyaldehyde (44 mg, 0.24 mmol) and 4-amino-2-phenyl-benzoic acid methyl ester (68 mg, 0.30 mmol) in CH₂Cl₂ (5 mL) was added TiCl₄ (0.3 mL) at 0° C. After stirring for 15 min, NaBCNH₃ (22 mg, 0.35 mmol) was added. The mixture was stirred at r.t. for overnight, and the product was extracted with CH₂Cl₂ (50 mL) from sat NaHCO₃ (20 mL). The organic layer was washed with brine and dried (MgSO₄). The crude product was purified by SiO₂ column chromatography with CHCl₃:acetone:EtOH=100:20:4 to afford the product as a white solid (59 mg, 62%). m.p. 155-156° C.; ¹H NMR (CDCl₃) 7.79 (d, J=8.5 Hz, 1H, Aryl H), 7.54 (s, 1H, imid-2H), 7.38-7.24 (m, 8H, Aryl H), 7.05 (s, 1H, imid-4H), 7.04-7.02 (m, 2H, Aryl H), 6.45 (dd, J=2.2 and 8.5 Hz, 1H, Aryl H), 6.35 (d, J=2.2 Hz, 1H, Aryl H), 5.13 (s, 2H, CH₂N), 4.23 (t, J=5.2 Hz, 1H, NH), 4.15 (d, J=5.2 Hz, 2H, CH₂NH), 3.58 (s, 3H, CO₂Me). Anal. calcd for C₂₅H₂₃N₃O₂.0.1H₂O: C, 75.20; H, 5.68; N, 10.53. Found: C, 75.03; H, 5.77; N, 10.46%.

(JJ-24).

To a solution of 4-(N-(1-(p-cyano)benzyl-1H-imidazol-5-yl)methyl)amino-2-phenylbenzoic acid (containing 39% w/w trifluoroacetic acid; 100 mg, 0.12 mmol, 0.34 mmol of trifluoroacetic acid), isopropylamine (7 mg, 0.12 mmol), triethylamine (47 mg, 0.46 mmol), and HOBt (32 mg, 0.24 mmol) in CH₂Cl₂ (1 mL) was added EDCI (23 mg, 0.12 mmol) at −10° C. The mixture was stirred overnight at r.t., and then diluted with CH₂Cl₂ (50 mL). The organic layer was washed with sat. NaHCO₃ (50 mL×2) and brine, and dried (MgSO₄). The crude product was purifed by SiO₂ column chromatography with CHCl₃:MeOH=10:1 to afford the product as a colorless amorphous (50 mg, 93%).

(JJ-25).

This compound was prepared by a similar method that described for JJ-24 by the reaction of 4-(N-(1-(p-cyano)benzyl-1H-imidazol-5-yl)methyl)amino-2-phenylbenzoic acid (containing 39% w/w trifluoroacetic acid; 100 mg, 0.12 mmol, 0.34 mmol of trifluoroacetic acid), cyclohexanemethylamine (14 mg, 0.12 mmol), triethylamine (47 mg, 0.46 mmol), HOBt (32 mg, 0.24 mmol), and EDCI (23 mg, 0.12 mmol) in CH₂Cl₂ (1 mL). The crude product was purified by SiO₂ column chromatography with CHCl₃:acetone:MeOH=100:40:8 to afford the desired product as a colorless amorphous (48 mg, 80%).

(JJ-26).

This compound was prepared by a similar method that described for JJ-24 by the reaction of 4-(N-(1-(p-cyano)benzyl-1H-imidazol-5-yl)methyl)amino-2-phenylbenzoic acid (containing 39% w/w trifluoroacetic acid; 100 mg, 0.12 mmol, 0.34 mmol of trifluoroacetic acid), benzylamine (13 mg, 0.12 mmol), triethylamine (47 mg, 0.46 mmol), HOBt (32 mg, 0.24 mmol), and EDCI (23 mg, 0.12 mmol) in CH₂Cl₂ (1 mL). The crude product was purified by SiO₂ column chromatography with CHCl₃:acetone:MeOH=100:40:8 to afford the desired product as a colorless amorphous (46 mg, 77%).

(JJ-35).

This compound was prepared by a similar method that described for JJ-24 by the reaction of 4-(N-(1-(p-cyano)benzyl-1H-imidazol-5-yl)methyl)amino-2-phenylbenzoic acid (containing 39% w/w trifluoroacetic acid; 100 mg, 0.12 mmol, 0.34 mmol of trifluoroacetic acid), 2-(ethylthio)ethylamine hydrochloride (17 mg, 0.12 mmol), triethylamine (59 mg, 0.58 mmol), HOBt (32 mg, 0.24 mmol), and EDCI (23 mg, 0.12 mmol) in CH₂Cl₂ (1 mL). The crude product was purified by SiO₂ column chromatography with CHCl₃:acetone:MeOH=100:40:8 to afford the desired product as a colorless amorphous (45 mg, 76%).

(JJ-36).

This compound was prepared by a similar method that described for JJ-24 by the reaction of 4-(N-(1-(p-cyano)benzyl-1H-imidazol-5-yl)methyl)amino-2-phenylbenzoic acid (containing 39% w/w trifluoroacetic acid; 100 mg, 0.12 mmol, 0.34 mmol of trifluoroacetic acid), 3-aminobenzonitrile (14 mg, 0.12 mmol), triethylamine (47 mg, 0.46 mmol), HOBt (32 mg, 0.24 mmol), and EDCI (23 mg, 0.12 mmol) in CH₂Cl₂ (1 mL). The crude product was purified by SiO₂ column chromatography with CHCl₃:acetone:MeOH=100:40:8 followed by Sephadex LH-20 gel chromatography with CHCl₃:MeOH=1:1 to afford the desired product as a brown solid (10 mg, 16%).

(JJ-37).

This compound was prepared by a similar procedure that described for JJ-20 by a reaction of 1-(p-phenyl)benzyl-5-imidazolecarboxyaldehyde (58 mg, 0.26 mmol) and 4-amino-2-phenyl-benzoic acicd methyl ester (59 mg, 0.26 mmol), TiCl₄ (25 mg, 0.13 mmol), and NaBCNH₃ (16 mg, 0.25 mmol). The crude product was isolated by SiO₂ column chromatography with CHCl₃:acetone:MeOH=100:40:8 to afford the desired product as a colorless amorphous (73 mg, 70%).

(JJ-73).

To a solution of 2-amino-biphenyl (11 mg, 0.17 mmol) and 1-(p-cyano)benzyl-5-imidazolecarboxyaldehyde (8 mg, 0.036 mmol) in MeOH (0.5 mL) was added AcOH (0.2 mL) and the mixture was stirred for 30 min at r.t. To the solution was added NaCNBH3 (1.4 mg, 0.04 mmol), and the mixture was stirred overnight. The product was extracted with CH₂Cl₂ (1 mL×3) from sat. NaCl, and the organic layer was passed through MgSO4 column. The solution was concentrated to give a pale pink amorphous. This crude product was purified by preparative TLC with CHCl₃:acetone:MeOH=100:40:8 to give the product (8 mg, 61%).

(JJ51)

p-NO₂-imid-BP-COOMe

Yellow oily solid, 52%.

¹NMR (CDCl₃): 8.11 (d, 2 H, ortho to NO₂), 7.79 (d, 1 H, ortho to COOMe), 7.60 (s, 1 H, NCHN), 7.33-7.37 (m, 3 H, aryl), 7.21-7.22 (dd, 2 H, aryl), 7.13 (d, 2 H, meta to NO₂), 7.12 (s, 1 H, NCHCCHO), 6.47 (dd, 1 H, para to biphenyl aryl), 6.34 (d, 1 H, ortho to biphenyl aryl), 5.28 (s, 2 H, NCH₂Ar), 4.19 (s, 1 H, NH), 4.17 (s, 2 H, NHCH₂), 3.59 (s, 3 H, COOCH₃).

(JJ52)

p-CH₃-imid-BP-COOMe

White amorphous solid, 43%.

¹NMR (CDCl₃): 7.79 (d, 1 H, ortho to COOMe), 79.52 (s, 1 H, NCHN), 7.31-7.37 (m, 3 H, aryl), 7.22-7.24 (dd, 2 H, aryl), 7.07 (d, 2 H, meta to CH₃), 7.02 (s, 1 H, NCHCCHO), 6.90 (d, 2 H, ortho to CH₃), 6.44 (dd, 1 H, para to biphenyl aryl), 6.29 (d, 1 H, ortho to biphenyl aryl), 5.07 (s, 2 H, NCH₂Ar), 4.1 (s, 1 H, NH), 4.14 (s, 2 H, NHCH₂), 3.57 (s, 3 H, COOCH₃), 2.28 (s, 3 H ArCH₃).

(JJ53)

p-Cl-imid-BP-COOMe

Clear oil, 62%.

¹NMR (CDCl₃): 7.79 (d, 1 H, ortho to COOMe), 7.51 (s, 1 H, NCHN), 7.31-7.37 (m, 4 H, aryl), 7.22-7.25 (m, 4 H, aryl and ortho to Cl), 7.01 (s, 1 H, NCHCCHO), 6.93 (d, 2 H, meta to Cl), 6.47 (dd, 1 H, para to biphenyl aryl), 6.36 (d, 1 H, ortho to biphenyl aryl), 5.10 (s, 2 H, NCH₂Ar), 4.66 (s, 1 H, NH), 4.12 (s, 2 H, NHCH₂), 3.57 (s, 3 H, COOCH₃).

(JJ57)

p-Br-imid-BP-COOMe

Pale yellow oil, 80%

¹H NMR (CDCl₃): 7.78 (d, 1 H, ortho to COOMe), 7.54 (s, 1 H, NCHN), 7.39 (d, 2 H, ortho to Br), 7.29-7.38 (m, 5 H, aryl), 7.23 (d, 2 H, meta to Br), 7.04 (s, 1 H, NCHCCH₂N), 6.48 (dd, 1 H, para to biphenyl aryl), 6.36 (d, 1 H, ortho to biphenyl aryl), 5.10 (s, 2 H, NCH₂Ar), 4.50 (s, 1 H, NH), 4.13 (s, 2 H, NHCH₂), 3.57 (s, 3 H, COOCH₃), ¹³C NMR (CDCl₃): 168.3 (C═O), 149.8 (para to COOMe), 145.6 (para to Br), 142.2 (biphenyl connection on COOMe ring), 134.9 (NCHN), 132.6 (ortho to COOMe), 132.2 (ortho to Br), 127.8-129.2 (biphenyl aryl, imidazole 4 position), 127.0 (meta to Br), 122.3 (imidazole 5 position), 118.8 (aryl connected to Br), 114.7 (ortho to biphenyl on COOMe ring), 110.9 (meta to biphenyl on COOMe ring), 51.4 (COOCH₃), 48.5 (CCH₂NHAr), 37.6 (NCH₂Ar),

(JJ58)

p-NH₂-imid-BP-COOMe

White amorphous solid, 40%.

¹H NMR (CDCl₃): 7.78 (d, 1 H, ortho to COOMe), 7.53 (s, 1 H, NCHN), 7.25-7.39 (m, 5 H, biphenyl aryl), 6.99 (s, 1 H, NCHCCH₂N), 6.84 (d, 2 H, meta to NH₂), 6.57 (d, 2 H, ortho to NH₂), 6.48 (dd, 1 H, para to biphenyl), 6.36 (d, 1 H, ortho to biphenyl), 5.00 (s, 2 H, NCH₂Ar), 4.53 (t, 1 H, CH₂NH), 4.16 (d, 2 H, CH₂NH), 3.63 (s, 3 H, COOCH₃).

(JJ59)

p-OMe-imid-BP-COOMe

Clear oil, 30%.

¹NMR (CDCl₃): 7.78 (d, 1 H, ortho to COOMe), 7.50 (s, 1 H, NCHN), 7.24-7.37 (m, 5 H, aryl), 7.00 (s, 1 H, NCHC), 6.95 (d, 2 H, meta to OMe), 6.78 (d, 2 H, ortho to OMe), 6.46 (dd, 1 H, para to biphenyl aryl), 6.34 (d, 1 H, ortho to biphenyl aryl), 5.04 (s, 2 H, NCH₂Ar), 4.36 (s, 1 H, NH), 4.14 (s, 2 H, NHCH₂), 3.72, (s, 3 H, OCH₃), 3.57 (s, 3 H, COOCH₃).

(JJ81)

m-NO₂-imid-BP-COOMe

Yellow amorphous solid, 37%. HRMS (FAB M+H): calcd. 443.171931 found 443.172100. ¹H NMR (CDCl₃): δ 8.09 (d, 1 H, J=10 Hz, Ar), 7.90 (s, 1 H, Ar), 7.75 (dd, 1 H, J=9 and 1 Hz, Ar), 7.56 (s, 1 H, imid.), 7.43 (td, 1 H, J=7 and 1 Hz, Ar), 7.27-7.37 (m, 5 H, biphenyl), 7.21 (d, 2 H, J=7 Hz, Ar), 7.07 (s, 1 H, imid.), 6.47 (dd, 1 H, J=10 and 3 Hz, Ar), 6.35 (d, 1 H, J=3 Hz, Ar), 5.26 (s, 2 H, CH₂Ar), 4.54 (t, 1 H, J=5 Hz, NH), 4.17 (d, 2 H, J=5 Hz, CH₂N), 3.57 (s, 3 H, COOCH₃).

(JS82)

m-CH₃-imid-BP-COOMe

White amorphous solid, 27%. HRMS (FAB, M+H): calcd. 412.202502 found 412.202400. ¹H NMR (CDCl₃): δ 7.77 (d, 1 H, J=8 Hz, Ar), 7.52 (s, 1 H, imid.), 7.21-7.36 (m, 5 H, biphenyl), 7.17 (t, 1 H, J=7 Hz, Ar), 7.08 (d, 1 H, J=10 Hz, Ar), 7.00 (s, 1 H, imid.), 6.81 (d, 2 H, J=7 Hz, Ar), 6.43 (dd, 1 H, J=3 and 8 Hz, Ar), 6.34 (d, 1 H, J=3 Hz, Ar), 5.07 (s, 2 H, CH₂Ar), 4.49 (t, 1 H, J=5.5 Hz, NH), 4.14 (d, 2 H, J=5.5 Hz, CH₂N), 3.55 (s, 3 H, COOCH₃).

(JJ83)

m-NH₂-imid-BP-COOMe

Yellow oily solid, 44%. HRMS (FAB, M+H): calcd. 413.197751 found 413.197500.

¹H NMR (CDCl₃): δ 7.79 (d, 1 H, J=9 Hz, Ar), 7.56 (s, 1 H, imid.), 7.24-7.38 (m, 5 H, biphenyl), 7.05-7.09 (m, 2 H, Ar), 6.58 (dd, 1 H, J=1 and 9 Hz, Ar), 6.41-6.45 (m, 2 H, Ar), 6.34 (d, 1 H, J=3 Hz, Ar), 6.23 (s, 1 H, Ar), 5.03 (s, 2 H, CH₂Ar), 4.17 (br. s, 2 H, CH₂N), 4.02 (br. s, 1 H, NH), 3.57 (s, 3 H, COOCH₃).

(JJ84)

m-Cl-imid-BP-COOMe

Off-White oily solid, 20%. HRMS (FAB, M+H): calcd. 432.147880 found 432.147800.

¹H NMR (CDCl₃): δ 7.79 (d, 1 H, J=9.5 Hz, Ar), 7.54 (s, 1 H, imid.), 7.18-7.38 (m, 7 H, Ar), 7.05 (s, 1 H, imid.), 7.01 (s, 1 H, Ar), 6.87 (d, 1 H, J=7 Hz, Ar), 6.47 (dd, 1 H, J=9.5 and 2 Hz, Ar), 6.38 (d, 1 H, 2 Hz), 5.11 (s, 2 H, CH₂Ar), 4.29 (br. s, 1 H, NH), 4.15 (br. s, 2 H, CH₂N), 3.57 (s, 3 H, COOCH₃).

(JJ85)

m-Br-imid-BP-COOMe

White oily solid, 20%. HRMS (FAB, M+H): calcd. 476.097363 found 476.097300.

¹H NMR (CDCl₃): δ 7.79 (d, 1 H, J=9 Hz, Ar), 7.56 (s, 1 H, imid.), 7.41 (d, 1 H, J=7 Hz, Ar), 7.23-7.38 (m, 5 H, biphenyl), 7.18 (s, 1 H, Ar), 7.14 (t, 1 H, J=9 Hz, Ar), 7.06 (s, 1 H, imid.), 6.93 (d, 1 H, J=7 Hz, Ar), 6.48 (dd, 1 H, J=9 and 3 Hz, Ar), 6.39 (d, 1 H, 3 Hz, Ar), 5.12 (s, 2 H, CH₂Ar), 4.35 (t, 1 H, J=5 Hz, NH), 4.16 (d, 2 H, J=5 Hz, CH₂N), 3.58 (s, 3 H, COOCH₃).

(JJ97)

m-CN-imid-BP-COOMe

Off-white oily solid, 41%. HRMS (FAB, M+H): calcd. 423.182101 found 423.182200.

¹H NMR (CDCl₃): δ 7.79 (d, 1 H, J=8.5 Hz, Ar), 7.55 (d, 2 H, J=7 Hz, Ar), 7.19-7.40 (m, 8 H, Ar), 7.09 (s, 1 H, imid.), 6.48 (dd, 1 H, J=2 and 8.5 Hz, Ar), 6.37 (d, 1 H, J=2 Hz, Ar), 5.19 (s, 2 H, CH₂Ar), 4.36 (t, 1 H, J=5 Hz, NH), 4.16 (d, 2 H, J=5 Hz, CH₂N), 3.58 (s, 3 H, COOCH₃).

(JJ101)

o-CH₃-imid-BP-COOMe

Yellow amorphous solid, 24%. HRMS (FAB, M+H): calcd. 412.202502 found 412.202400. ¹H NMR (CDCl₃): δ 7.79 (d, 1 H, J=9 Hz, Ar), 7.47 (s, 1 H, imid.), 7.33-7.37 (m, 3 H, Ar), 7.10-7.25 (m, 6 H, Ar), 6.69 (d, 1 H, J=S Hz, Ar), 6.45 (dd, 1 H, J=9 and 2 Hz, Ar), 6.34 (d, 1 H, J=2 Hz, Ar), 5.11 (s, 2 H, CH₂Ar), 4.19 (d, 2 H, J=6 Hz, CH₂N), 3.89 (t, 1 H, J=6 Hz, NH), 3.59 (s, 3 H, COOCH₃).

(JJ102)

o-Br-imid-BP-COOMe

Yellow oil, 24%. HRMS (FAB, M+H): calcd. 476.097363 found 476.097300.

¹H NMR (CDCl₃): δ 7.77 (d, 1 H, J=9 Hz, Ar), 7.54 (d, 1 H, J=9 Hz, Ar), 7.49 (s, 1 H, imid.), 7.29-7.37 (m, 3 H, Ar), 7.22-7.26 (m, 2 H, Ar), 7.12-7.19 (m, 2 H, Ar), 7.07 (s, 1 H, imid.), 6.63 (d, 1 H, J=7 Hz, Ar), 6.47 (dd, 1 H, J=2 and 9 Hz, Ar), 6.34 (d, 1 H, J=2 Hz, Ar), 5.19 (s, 2 H, CH₂Ar), 4.34 (t, 1 H, J=6 Hz, NH), 4.19 (d, 2 H, J=6 Hz, CH₂N), 3.57 (s, 3 H, COOCH₃).

(JJ103)

o-CN-imid-BP-COOMe

Clear oily solid, 47%. HRMS (FAB, M+H): calcd. 423.182101 found 423.182200.

¹H NMR (CDCl₃): δ 7.78 (d, 1 H, J=8 Hz, Ar), 7.64 (dd, 1 H, J=8 and 2 Hz, Ar), 7.53 (s, 1 H, imid.), 7.48 (td, 1 H, J=8 and 2 Hz, Ar), 7.32-7.41 (m, 4 H, Ar), 7.22-7.27 (m, 2 H, Ar), 7.11 (s, 1 H, imid.), 6.91 (d, 1 H, J=8 Hz, Ar), 6.50 (dd, 1 H, J=3 and 8 H, Ar), 6.37 (d, 1 H, J=3 Hz, Ar), 5.37 (s, 2 H, CH₂Ar), 4.27 (t, 1 H, J=5 Hz, NH), 4.24 (d, 2 H, J=5 Hz, CH₂N), 3.58 (s, 3 H, COOCH₃).

(JJ105)

o-NH₂-imid-BP-COOMe

Yellow oil, 59%. HRMS (FAB, M+H): calcd. 413.197751 found 413.197900.

¹H NMR (CDCl₃): δ 7.78 (d, 1 H, J=8 Hz, Ar), 7.42 (br. s, 1 H, imid), 7.35-7.38 (m, 3 H, Ar), 7.22-7.25 (m, 2 H, Ar), 7.12 (td, 1 H, J=7 and 1 Hz, Ar), 7.01 (s, 1 H, imid.), 6.66-6.76 (m, 3 H, Ar), 6.52 (dd, 1 H, J=2 and 8 Hz, Ar), 6.42 (d, 1 H, J=2 Hz, Ar), 4.98 (s, 2 H, CH₂Ar), 4.20 (br. s, 2 H, CH₂N), 3.57 (s, 3 H, COOCH₃).

(JJ118)

p-NO₂-imid-BP

Yellow amorphous solid, 77%. HMS (FAB M+H): calcd. 3850166451 found 385.166500. ¹H NMR (CDCl₃): δ 8.16 (d, 2 H, J=8 Hz, ortho to NO₂), 7.62 (s, 1 H, imid), 7.50 (d, 2 H, J=7 Hz, aryl), 7.42 (t, 2 H, J=7 Hz, aryl), 7.34 (d, 1 H, J=7 Hz, aryl), 7.22 (d, 1 H, J=9 Hz, aryl), 7.17, (d, 2 H, J=8 Hz, meta to NO₂), 7.00 (d, 1 H, J=9 Hz, aryl), 6.72 (s, 1 H, aryl), 6.52 (d, 1 H, J=9 Hz, aryl), 5.34 (s, 2 H, CH₂Ar), 4.17 (s, 2 H, CH₂N).

JJ119

p-CH₃-imid-BP

Yellow amorphous solid, 28%. HRMS (FAB M+H): calcd. 354.197023 found 354.196900. ¹H NMR (CDCl₃): δ 752-7.58 (m, 3 H, aryl), 7.29-7.43 (m, 5 H, aryl), 7.09-7.15 (m, 3 H, aryl), 6.96-6.98 (m, 2 H, aryl), 6.70 (t, 1 H, J=1 Hz, aryl), 6.53 (dd, 1 H, J=1 and 8 Hz, aryl), 5.16 (s, 2 H, CH₂Ar), 4.18 (s, 2 H, CH₂N), 2.33 (s, 3 H, CH₃).

JJ120

p-Cl-imid-BP

Yellow oil, 45%. HRMS (FAB M+H): calcd. 374.142401 found 374.142400. ¹H NMR (CDCl₃): δ 7.51 (d, 3 H, J=7 Hz, aryl), 7.40 (t, 2 H, J=8 Hz, aryl), 7.21-2.34 (m, 5 H, aryl), 7.06 (s, 1 H, imid), 6.97 (t, 3 H, J=7 Hz, aryl), 6.71 (bt, 1 H. J=1 Hz, aryl), 6.52 (dd, 1 H, J=1 and 8 Hz, aryl), 5.12 (s, 2 H, CH₂Ar), 4.12 (s, 2 H, CH₂N).

JJ121

p-Ph-imid-BP

off-white solid, 73%. HRMS (FAB M+H): calcd. 416.212673 found 416.212800. ¹H NMR (CDCl₃): δ 7.63 (s, 1 H, aryl), 7.53-7.57 (m, 5 H, aryl), 7.49-7.51 (m, 2 H, aryl), 7.29-7.46 (m, 7 H, aryl), 7.12-7.14 (m, 3 H, aryl), 6.98 (td, 1 H, J=2 and 1 Hz, aryl), 6.55 (dd, 1 H, J=1 and 8 Hz, aryl), 5.25 (s, 2 H, CH₂Ar), 4.20 (s, 2 H, CH₂N).

V-72 (JJ128)

White amorphous solid, 26%

HRMS (FAB M+H): calcd. 488.233803, found 488.233900 7.79 (d, J=8 Hz, 1 H, ortho to CONH), 7.64 (s, 1 H, imidazole), 7.49-7.53 (m, 5 H), 7.42-7.45 (m, 2 H), 7.29-7.40 (m, 5 H), 7.22-7.23 (m, 2 H), 7.08-7.12 (m, 3 H), 6.46 (dd, J=8 Hz and 3 Hz, 1 H, ortho to NH), 6.33 (d, J=3 Hz, 1 H, ortho to NH), 5.20 (s, 2 H, CH₂Ar), 4.20 (d, 6 Hz, 2 H, CH₂NH), 4.02 (q, J=7 Hz, 2 H, COOCH₂CH₃), 3.84 (bt, J=6 Hz, 1 H, CH₂NH), 0.97 (t, J=7 Hz, 3 H, COOCH₂CH₃)

V-74 (JJ129)

Yellowish amorphous solid, 21%

HRMS (FAB M+H): calcd. 502.249453, found 502.249700 7.75 (d, J=8 Hz, 1 H, ortho to COOPr), 7.63 (s, 1 H, imidazole), 7.46-7.51 (m, 4 H), 7.39-7.42 (m, 2 H), 7.27-7.36 (m 6 H), 7.19-7.22 (m, 2 H), 7.07-7.10 (m, 3 H), 6.44 (dd, J=8 Hz and 2 Hz, 1 H, ortho to NH), 6.30 (d, J=2 Hz, 1 H, ortho to NH), 5.17 (s, 2 H, CH₂Ar), 4.88 (p, J=6 Hz, 1 H, CH(CH₃)₂), 4.17 (s, 2 H, CH₂NH), 0.95 (d, J=6 Hz, 6 H, CH(CH₃)₂)

VIII-44a (JJ130)

Yellow amorphous solid, 20%

HRMS (FAB M+H): calcd. 542.280753, found 542.280700 7.80 (d, J=9 Hz, 1 H), 7.55 (s, 1 H, imidazole), 7.49 (d, J=8 Hz, 5 H), 7.41 (t, J=8 Hz, 3 H), 7.33-7.37 (m, 1 H), 7.28-7.31 (m, 3 H), 7.21-7.25 (m, 2 H), 7.04-7.08 (m, 3 H), 6.47 (dd, J=9 Hz and 3 Hz, ortho to NH), 6.32 (d, J=3 Hz, ortho to NH), 5.14 (s, 2 H, CH₂Ar), 4.69 (m, 1 H, COOCH), 4.30 (m, 1 H, CH₂NH), 4.15 (d, J=5 Hz, CH₂NH), 1.62-1.66 (m, 2 H), 1.51-1.53 (m, 2 H), 1.41-1.44 (m, 1 H), 1.09-1.27 (m 5 H).

VI-54b (JJ136)

Clear oil, 39%

HRMS (FAB M+H): calcd. 499.195644, found 499195600. 7.62 (s, 1 H, imidazole), 7.46-7.52 (m, 5 H), 7.41 (t, J=8 Hz, 2 H), 7.34-7.36 (m, 1 H), 7.26-7.27 (m, 2 H), 7.22-7.75 (m, 2 H), 7.31 (d, J=8 Hz, 1 H), 7.10-7.16 (m, 5 H), 6.54 (dd, J=4 Hz and 8 Hz, 1 H, ortho to NH), 6.46 (d, J=4 Hz, 1 H, ortho to NH), 5.22 (s, 2 H, CH₂Ar), 4.18 (d, J=5 Hz, 2 H, CH₂NH)

VI-62 (JJ137)

Black amorphous solid, 13%

HRMS (FAB M+H): calcd. 481.239222, found 481.239100 7.67 (s, 1 H), 7.51-7.53 (m, 6 H), 7.36-7.45 (m, 7 H), 7.30-7.31 (m, 4 1), 7.23-7.24 (m, 2 H), 7.11-7.15 (m, 5 H), 6.55 (dd, J=3 Hz and 9 Hz, 1 H, ortho to NH), 6.50 (m, 1 H), 6.44 (d, J=3 Hz, 1 H, ortho to NH), 6.15 (m, 1 H), 6.11-6.13 (m, 1 H), 5.24 (s, 2 H, CH₂Ar), 4.19 (bs, 2 H, CH₂NH)

VI-76 (JJ138)

Yellow amorphous solid, 21%

HRMS (FAB M+H): calcd. 532.238888, found 532.239000 7.76 (d, J=8 Hz, 1 H), 7.65 (s, 1 H, imidazole), 7.50-7.55 (m, 5 H), 7.41-7.45 (m, 3 H), 7.33-7.40 (m, 7 H), 7.09-7.18 (m, 6 H), 6.61 (dd, J=3 Hz and 8 Hz, 1 H ortho to NH), 6.43 (d, J=3 Hz, 1 H, ortho to NH), 5.71 (d, J=1 Hz, 1 H), 5.24 (s, 2 H, CH₂Ar), 4.21 (d, J=5 Hz, 2 H, CH₂NH), 3.72 (m, 1 H, CH₂NH)

VI-78 (JJ139)

Tan solid, 12%

HRMS (FAB M+H): calcd. 549.211294, found 549.209200 7.98 (dd, J=10 Hz and 13 Hz, 2 H), 7.65 (d, 8 Hz, 2 H), 7.52 (t, J=10 Hz, 5 H), 7.28-7.45 (m, 10 H), 7.13 (d, J=8 Hz, 2 H), 6.60 (bd, J=9 Hz, 1 H, ortho to NH), 6.43 (bs, 1 H, ortho to NM, 5.23 (s, 2 H, CH₂Ar), 4.23 (d, J=2 Hz, 2 H, CH₂NH), 3.82 (m, 1 H, CH₂NH)

VI-102 (JJ140)

Off-white solid, 4%

HRMS (FAB M+H): calcd. 533.234137, found 533.234300

VI-112 (JJ141)

Yellow amorphous, 45%

HRMS (FAB M+H): calcd. 533.234137, found 533.234300 7.64 (d, J=1 Hz, 1 H), 7.50-7.56 (m, 4 H), 7.40-7.46 (m, 5 H), 7.33-7.37 (m, 1 H), 7.18-7.28 (m, 3 H) 7.14 (m, 3 H), 6.98 (d, J=9 Hz, 1 H), 6.88 (td, J=1 Hz and 8 Hz, 1 H), 6.54-6.58 (m, 3 H), 5.26 (s, 2 H, CH₂Ar), 4.19 (d, J=5 Hz, 2 H, CH₂NH), 3.61 (t, J=5 Hz, 1 H, CH₂NH)

VII-184 (JJ142)

Yellow amorphous, 33%

HRMS (FAB M+H): calcd. 533.234137 found 535.250000 7.79 (d, J=9 Hz, 1 H), 7.64 (s, 1 H, imidazole), 7.51 (m, 5 H), 7.43 (t, J=7 Hz, 2 H), 7.34-7.36 (m, 4 H), 7.28-7.32 (m, 4 H), 7.08-7.11 (m, 3 H), 7.00 (s, 1 H, imidazole), 6.48 (dd, J=3 Hz and 9 Hz, 1 H, ortho to NH), 6.33 (d, J=3 H, 1 H, ortho to NH), 5.20 (s, 2 H, CH₂Ar), 4.19 (d, J=5 Hz, 2 H, CH₂NH)

Biological Data

Compounds which appear in Table 1, below were tested in vivo using the Tulahuen strain of T. cruzi. See Buckner, et al., Antimicrobial Agents and Chemotherapy, 40, 2592-2597 (1996). In this assay, trypomastigotes were grown on monolayers of mouse 3T3 fibroblasts as previously described by Van Voorhis, et al., J. Exp. Med. 169: 641-652 (1989). The drug concentrations in the assay which resulted in 50% inhibition of T. cruzi growth on 3T3 fibroblasts appear in Table 1, below. The assay also tested for inhibition of fibroblast growth (an indication of potential toxicity). In virtually all instances, the compounds were non-toxic in the assay. The results of the assay for all compounds tested appear below in Table 1. The following structure identifies those compounds tested and set forth in Table 1, below. In Table 1, if a substituent is left blank, such a substituent is defined as a hydrogen.

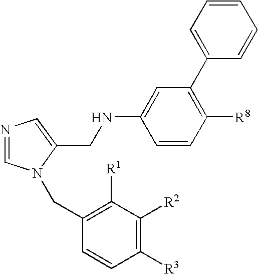

### CONCLUSIONS

Few trends were evident from the collected data, although in general, hydrophobic substitution showed better activity than more polar ones and para substitution resulted in more potency than meta or ortho. The most potent compound was the p-phenyl compound JJ37, with a remarkable activity of 500 pM. To our knowledge, this is among the most potent known compounds against T. cruzi amastigotes.

The compounds in the above class, JJ20-JJ105, all contain benzoate esters or amides. The free benzoic acid of the para-cyano compound, however, showed much decreased activity compared to its methyl ester: 1 μM for JJ28 compared with 40 nM for JJ20. Esterases and proteases are prevalent both extracellularly and within cells, especially in mice. Consequently a new scaffold was required to overcome this disadvantage.

TABLE 1

| Com- | | | | | EC₅₀ | EC₅₀ |
|---|---|---|---|---|---|---|
| pound | R⁸ | R³ | R² | R¹ | T. cruzi | Fibroblast |
| JJ20 | COOMe | CN | | | .04 | >10 |
| JJ21 | COOMe | | | | .08 | >10 |
| JJ24 | CONHiPr | CN | | | | |
| JJ25 | CONHCH₂Cy | CN | | | | |
| JJ26 | CONHBn | CN | | | | |
| JJ28 | COOH | CN | | | 1.0 | >10 |
| JJ35 | CONH(CH₂)₂SEt | CN | | | | |
| JJ36 | CONH(3-CN-Ph) | CN | | | | |
| JJ51 | COOMe | NO₂ | | | .1 | >10 |
| JJ52 | COOMe | CH₃ | | | .005 | >10 |
| JJ53 | COOMe | Cl | | | .005 | >10 |
| JJ57 | COOMe | Br | | | .02 | >10 |
| JJ58 | COOMe | NH₂ | | | .25 | >10 |
| JJ59 | COOMe | OCH₃ | | | .025 | >10 |
| JJ37 | COOMe | Ph | | | .0005 | >10 |
| JJ97 | COOMe | | CN | | .1 | >10 |
| JJ81 | COOMe | | NO₂ | | .03 | >10 |
| JJ82 | COOMe | | CH₃ | | .02 | >10 |
| JJ84 | COOMe | | Cl | | .02 | >10 |
| JJ85 | COOMe | | Br | | .02 | >10 |
| JJ83 | COOMe | | NH₂ | | .13 | >10 |
| JJ104 | COOMe | | CF₃ | | .04 | >10 |
| JJ103 | COOMe | | | CN | .2 | >10 |
| JJ101 | COOMe | | | CH₃ | 1.0 | 10 |
| JJ117 | COOMe | | | Cl | <0.1, | >10 |
| | | | | | >.01 | |
| JJ102 | COOMe | | | Br | .1 | 10 |
| JJ105 | COOMe | | | NH₂ | .005 | >10 |
| JJ73 | | CN | | | .05 | 10 |
| JJ118 | | NO₂ | | | .1 | 10 |
| JJ119 | | CH₃ | | | .1 | 10 |
| JJ120 | | Cl | | | .08 | 10 |
| JJ121 | | Ph | | | .01 | 10 |
| JJ128 | COOEt | Ph | | | .01 | >10 |
| JJ129 | COOiPr | Ph | | | .05 | >10 |
| JJ130 | COOCy | Ph | | | .01 | >10 |
| JJ136 | 2-pyrrole | Ph | | | .1 | >10 |
| JJ137 | 5-thiazole | Ph | | | .05 | >10 |
| JJ138 | 2-benzofuran | Ph | | | .1 | >10 |
| JJ139 | benzthiazole | Ph | | | .02 | >10 |
| JJ140 | benzoxazole | Ph | | | .04 | >10 |
| JJ141 | benzisoxazole | Ph | | | .01 | >10 |
| JJ142 | anthranil | Ph | | | .05 | >10 |

It was apparent from JJ20-JJ105 that the 4-phenybenzylimidazole moiety gave the best activity against T. cruzi, and so it was used in almost all of the compounds containg the new scaffold. A new scaffold was required which fulfilled the following criteria: hydrophobicity, stability, diversity, accessibility. The molecule must be largely hydrophobic, as previous SAR has suggested that the ester moiety only serves a steric role to enhance binding affinity or activity. The new scaffold must be stable enough for animal studies to be run, as it was found that the free acid was inactive against the parasite, presubably due to inability to permeate cells. The new scaffold must accomodate diversity, as SAR work was desired. Lastly, the molecules should be synthetically accessible. As a number of compounds with appropriate diversity is required a complex synthesis is undesirable.

It came as a surprise that removing the ester group of the original compound library had only a small effect on the compounds' activity. The aminobiphenyl compounds, JJ73 and JJ118-JJ121 bad generally only a 10-fold reduction in activity from their methyl ester-containing parent compounds. As with the ester-containing series the most potent compound was that containing a 4-phenylbenzylimidazole; JJ121 (IC₅₀=10 nM) had 20-fold reduced activity compared with JJ37 (500 pM). Also interesting is that the removal of the ester group significantly compressed the activity range of the tested compounds. The ester containing series varied almost 1000-fold whereas the aminobiphenyls only show a 10-fold activity range.

Varying the ester substituent showed a similar drop in activity as altogether eliminating the ester. All compounds tested had an IC₅₀ value of 10-50 nM, and there was no discernable trend in activity. The methyl ester remained 20 times more active than the others, even those such as the ethyl ester with little structural difference. All compounds tested had significantly higher activity than the analogous compounds containing a methionine methyl ester.

The clear advantage of the aminobiphenyl compounds has been shown to be in animal studies. The blood serum esterase levels of mice is very high, and it was thought that eliminating the ester group would increase the bioavailability of the aminobiphenyl over the ester. Interestingly, while all of the other esters were hydrolized very rapidly (within 30 minutes) to their benzoic acids the methyl ester shows much higher serum stability; the ester is the only species of JJ37 found after 30 minutes of incubation in murine serum but its fate after longer times remains unclear.

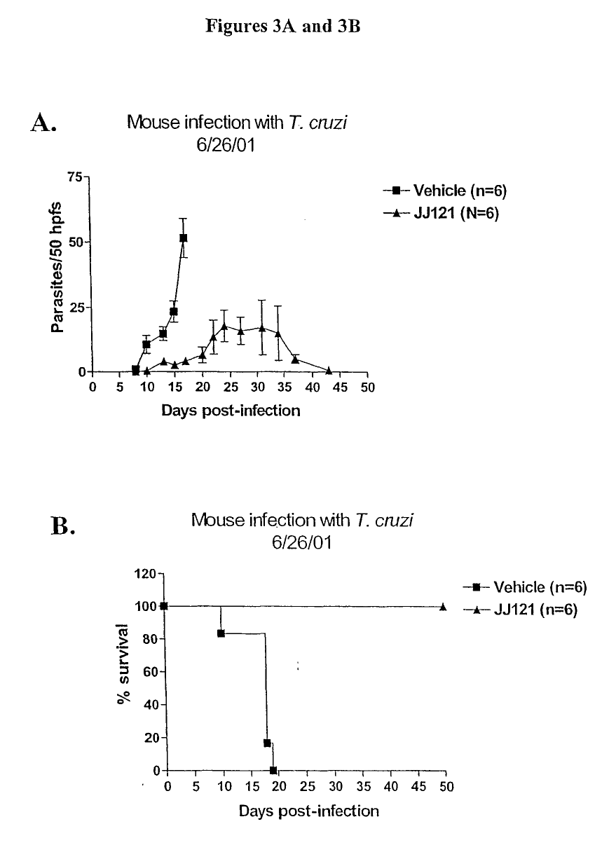

Despite this apparent stability, however, the results for anti-T. cruzi activity in infected mice is much better for JJ121 than for JJ37. JJ121 causes a dramatic suppression of parasite from mouse blood within 45 days at twice daily 50 mg/kg doses, whereas the level rises to fatal levels in control mice within 10-15 days. See FIGS. 3A and 3B. All mice receiving JJ121 survive past 100 days, whereas the vehicle mice all die by day 20. JJ37 shows activity in mouse models, but much less than that of JJ121.

All of the heteroterphenyl compounds tested show reasonably good activity against T. cruzi amastigotes. Like all of the compounds tested they do not show as strong of activity as JJ37; ED₅₀=10-100 nM for all compounds. It was again surprising that the compounds showed such little variance in activity. As their activity is in the same range as that for the aminobiphenyl series of compounds it appears that the heterocycle contributes little to the binding interaction that causes inhibitory activity.

The activity data suggest that the phenylbenzylimidazole dominates the interaction between these classes of compounds and the P-450_{14DM} whose inhibition results in antiparastic activity. Other than the methyl ester, the original lead compound whose picomolar inhibition is ≧20-fold more potent than any other compounds, all compounds screened have an ED₅₀ value of 10-100 nM.

Candida Assays

The compound JJ119 was tested for anti-Candida activity in a standard assay, as described below, against a number of strains of fungus. The assay compared inhibition (the effective dose causing 80% growth inhibition of the fungus) of several strains of Candida spp. using Fluconazole and compound JJ 119 (R³ is CH₃, all other variable substituents are H) of the present invention (see Table I, above). As set forth in Table II, below, the present compound exhibits favorable anti-Candida activity against a number of strains of Candida spp. Compounds JJ120 and JJ80 also exhibited activity in the assay.

The Candida assay followed the “Reference method for broth dilution antifungal susceptibility testing of yeasts; approved standard”, NCCLS, June, 1997. The procedure was modified to a 96 well format (as described in Modifications section 3.8). The methodology employed deviated from the approved method by reading results at 24 h rather than 48 h (since cultures of the ATCC strains were generally dense by 24 h).

In general, Candida were inoculated at a density of 2×10³/ml in RPMI based medium in the presence of drugs at the following concentrations: 50 uM, 10 uM, 2 uM, 0.4 uM, and 0 uM. Cells were incubated at 34° C. for 24 hr and growth inhibition was scored visually for turbidity. All drugs were diluted in DMSO (except fluconazole which was diluted in water). Top concentration of DMSO in the cultures was 0.25%; DMSO alone at 0.5% was not inhibitory to C. albicans.

TABLE II

| | ED80* | | | | | |
|---|---|---|---|---|---|---|
| Strain of Fungus | Fluconazole** | | | | JJ119*** | |
| C. albicans | 6.53 | | 6.53 | | 12.5 | 12.5 |
| strain 1 | | | | | | |
| C. albicans | >208.9 | (64) | >208.9 | (64) | 12.5 | 12.5 |
| strain 2 | | | | | | |
| C. albicans | <0.816 | (0.125) | <0.816 | (0.125) | 6.25 | 6.25 |
| strain 3 | | | | | | |
| C. krusei | 104.5 | (32) | 104.25 | (32) | 25 | 25 |
| strain 1 | | | | | | |
| C. krusei | 52.23 | (16) | 52.23 | (26) | 50 | 50 |
| strain 2 | | | | | | |
| C. krusei | 52.23 | (16) | 52.23 | (16) | 25 | 25 |
| strain 3 | | | | | | |
| C. glabrata | | | 104.5 | (32) | | 12.5 |
| strain 1 | | | | | | |
| C. glabrata | | | 6.528 | (2) | | 6.25 |
| strain 2 | | | | | | |
| C. glabrata | | | 208.9 | (64) | | 25 |
| strain 3 | | | | | | |
| C. tropicalis | >208.9 | (64) | >208.9 | (64) | >100 | >100 |
| strain 1 | | | | | | |
| C. tropicalis | >208.9 | (64) | >208.9 | (64) | >100 | >100 |
| strain 2 | | | | | | |
| C. tropicalis | >208.9 | (64) | >208.9 | (64) | >100 | >100 |
| strain 2 | | | | | | |
| C. parapsilosis | 1.632 | (0.5) | 1.632 | (0.5) | 100 | 50 |
| strain 1 | | | | | | |
| C. parapsilosis | <0.816 | (0.125) | <0.816 | (0.125) | 25 | 12.5 |
| strain 2 | | | | | | |

*Effective dose that causes 80% growth inhibition
**Expressed in μM (μg/ml)
***Expressed in μM

It is to be understood by those skilled in the art that the foregoing description and examples are illustrative of practicing the present invention, but are in no way limiting. Variations of the detail presented herein may be made without departing from the spirit and scope of the present invention as defined by the following claims.

### REFERENCES

1) Bastien, J. W. The Kiss of Death: Chagas' Disease in the Americas; University of Utah Press: Salt Lake City, 1998.

2) Urbina, J. A. J. Mol. Med. 1999, 77, 332.

3) Brener, Z. Trypanosoma cruzi: Taxonomy, Morphology and Lfe Cycle; Wendel, S., Brener, Z., Camargo, M. E. and Rassi, A., Ed.; International Society of Blood Transfusion: São Paulo, Brazil, 1992, pp 13-30.

4) Dias, J. C. P. Epidemiology of Chagas Disease; Wendel, S., Brener, Z., Camargo, M. E. and Rassi, A., Ed.; International Society of Blood Transfusion: São Paulo, Brazil, 1992, pp 49-80.

5) Rassi, A.; Luquetti, A. O.; Jr., A. R.; Rassi, S. G.; Rassi, A. G. Chagas Disease Clinical Features; Wendel, S., Brener, Z., Camargo, M. E. and Rassi, A., Ed.; International Society of Blood Transfusion: São Paulo, Brazil, 1992, pp 81-102.

6) Barrett-Bee, K.; Ryder, N. Biochemical Aspects of Ergosterol Biosynthesis Inhibition; Joyce A. Sutcliffe, N. H. G., Ed.; Chapman and Hall: New York, 1992, pp 410.

7) Yoshida, Y.; Aoyama, Y. J. Biol. Chem. 1984, 259, 1655.

8) Aoyama, Y.; Yoshida, Y.; Sato, R. J. Biol. Chem. 1984, 259, 1661.

9) Aoyama, Y.; Ysida, Y.; Sonoda, Y.; Sato, Y. J. Biol. Chem. 1987,262, 1239.

10) Aoyama, Y.; Ysida, Y.; Sonoda, Y.; Sato, Y. J. Biol. Chem. 1989, 264, 18502.

11) Ortiz de Montellano, P. R. Oxygen Activation and Reactivity; Second ed.; Montellano, P. R. O. d., Ed.; Plenum Press: New York, 1995.
12) Oehlschlager, A. C.; Czyzewska, E. Rationally Designed Inhibitors of Sterol Biosynthesis; Joyce A. Sutcliffe, N. H. G., Ed.; Chapman and Hall: New York, 1992, pp 410.

13) Yoshida, Y.; Aoyama, Y. Biochem. Soc. Trans. 1991, 19, 778.

14) Hitchcock, C. A. Biochem. Soc. Trans. 1991, 19, 782.

15) Podust, L. M.; Poulous, T. L.; Waterman, M. R. Proc. Nat. Acad. Sci. U.S.A. 2001, 98, 3068-3073.

16) Urbina, J. A.; Payares, G.; Molina, J.; Sanoja, C.; Liendo, A.; Lazardi, K.; Piras, M. M.; Piras, R.; Perez, N.; Wincker, P.; Ryley, J. F. Science 1996, 273, 969.

17) Urbina, J. A. Parasitology 1997, 114, S91.

18) Urbina, J. A.; Paares, G.; Contreras, L. M.; Liendo, A.; Sanoja, C.; Molina, J.; Piras, M.; Piras, R.; Perez, N.; Wincker, P.; Loebenberg, D. Antimicrob. Agents Chemother. 1998, 42, 1771.

19) Urbina, J. A.; Lira, R.; Visbal, G.; Bartoli, J. Antimicrob. Agents Chemother. 2000, 44, 2498-2502.

20) Yokoyama, K.; Trobridge, P.; Buckner, F. S.; Voorhis, W. C. V.; Stuart, K. D.; Gelb, M. H. J. Biol. Chem. 1998, 273, 26497.

21) Ohkanda, J.; Lockmnan, J. W.; Yokoyama, K.; Gelb, M. H.; Croft, S. L.; Kendrick, H.; Harrell, M. I.; Feagin, J. E.; Blaskovich, M. A.; Sebti, S. M.; Hamilton, A. D. Bioorg. Med. Chem. Lett. 2001, 11, 761-764.

22) Gelb, M. H., Personal Communication.

23) Sommerburg, O.; Zang, L.-Y.; van Kuijik, F. J. G. M. J. Chromotog. B 1997, 695, 209-215.

1. A compound according to formula I: 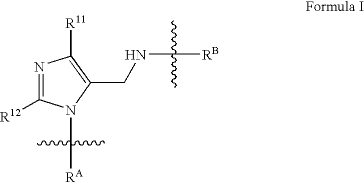
Where R^{A} is a C₁-C₁₀ substituted or unsubstituted linear, branch-chained or cyclic alkyl or alkenyl group or a phenyl group according to the formula: 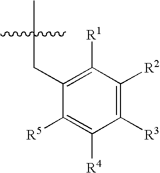
R^{B} is a C₁-C₁₀ substituted or unsubstituted linear, branch-chained or cyclic alkyl or alkenyl group or a phenyl group of the formula: 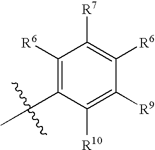
R¹, R², R³, R⁴, R⁵, R⁶, R⁷, R⁸, R⁹ and R¹⁰ are each independently selected from H, a C₁-C₁₀ alkyl or alkenyl group, CF₃, F, Cl, Br, I, CN, NO₂, NH₂, NHR, NRR, COR, OR, CO₂R,
or COSR, where R is H or a C₁-C₁₀ alkyl or alkenyl group, an unsubstituted or substituted aryl or heterocycle group,
or a 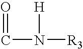
group, where R₃ is H, a C₁-C₁₀ alkyl, alkenyl, ether or a thioether group; and
R¹¹ and R¹² are independently selected from H or a C₁-C₃ alkyl or alkenyl group,
or a pharmaceutically acceptable salt thereof.

2. The compound according to claim 1 wherein R^{A} and R^{B} are substituted phenyl groups, R¹, R², R³, R⁴, R⁵, R⁶, R⁷, R⁸, R⁹ and R¹⁰ are independently H or a C₁-C₃ alkyl group, CO₂R, OR, CN, CF₃, Cl, Br, NRR, NO₂, unsubstituted phenyl and R is H or CH₃ .

3. The compound according to claim 1 wherein R^{A} and R^{B} are substituted phenyl groups, R¹, R² and R³ are independently selected from H, Phenyl, CN, CF₃, Cl, Br, OR, NRR, NO₂ and CH₃, R⁴, R⁵, R⁶, R⁹, R¹⁰, R¹¹ and R¹² are each H, R⁷ is phenyl or substituted phenyl, R⁸ is selected from H, CN, CF₃, Cl, Br, OR, NRR, NO₂, CH₃ and COOR and R is H or C₁-C₃ alkyl.

4. The compound according to claim 3 wherein when one of R¹, R² and R³ is other than H, the other of R¹, R² or R³ are H and R is H or CH₃.

5. The compound according to claim 4 wherein R¹ is NH₂, CN or Br, R⁷ is phenyl and R⁸ is COOR.

6. The compound according to claim 5 wherein R² and R³ are both H.

7. The compound according to 6 wherein R¹ is NH₂ and R is CH₃.

8. The compound according to claim 3 wherein R⁷ is phenyl, R⁸ is COOR and R is a C₁-C₃ alkyl.

9. The compound according to claim 8 wherein R¹ and R² are H, R³ is H, CN, phenyl, CH₃, OCH₃, Br or Cl and R is CH₃.

10. The compound according to claim 9 wherein R³ is phenyl, Cl or CH₃.

11. The compound according to claim 10 wherein R³ is phenyl.

12. The compound according to claim 3 wherein R¹ and R³ are H, R² is CN, NO₂, CH₃, Cl, Br or CF₃, R⁷ is phenyl, and R is C₁-C₃ alkyl.

13. The compound according to claim 12 wherein R² is CH₃, Cl or Br and R is CH₃.

14. The compound according to claim 3 wherein R¹ and R² are H, R³ is CN, NO₂, CH₃, Cl, Phenyl, Br or CF₃, R⁷ is phenyl and R⁸ is H.

15. The compound according to claim 14 wherein R³ is CN or phenyl.

16. The compound according to claim 14 wherein R³ is phenyl.

17. A pharmaceutical composition comprising an effective amount of a compound according to formula I: 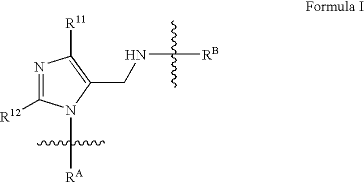
Where R^{A} is a C₁-C₁₀ substituted or unsubstituted linear, branch-chained or cyclic alkyl or alkenyl group or a phenyl group according to the formula: 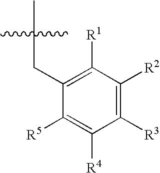
R^{B} is a C₁-C₁₀ substituted or unsubstituted linear, branch-chained or cyclic alkyl or alkenyl group or a phenyl group of the formula: 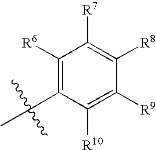
R¹, R², R³, R⁴, R⁵, R⁶, R⁷, R⁸, R⁹ and R¹⁰ are each independently selected from H, C₁-C₁₀ alkyl or alkenyl group, CF₃, F, Cl, Br, I, CN, NO₂, NH₂, NHR, NRR, COR, OR, CO₂R,
or COSR, where R is H or a C₁-C₁₀ alkyl or alkenyl group, an unsubstituted or substituted aryl or heterocycle group,
or a 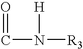
group, where R₃ is H, a C₁-C₁₀ alkyl, alkenyl, ether or a thioether group; and
R¹¹ and R² are independently selected from H or a C₁-C₃ alkyl or alkenyl group,
or a pharmaceutically acceptable salt thereof,
optionally in combination with a pharmaceutically acceptable additive carrier or excipient.

18. The composition according to claim 17 wherein R^{A} and R^{B} are substituted phenyl groups, R¹, R², R³, R⁴, R⁵, R⁶, R⁷, R⁸, R⁹ and R¹⁰ are independently H or a C₁-C₃ alkyl group, CO₂R, OR, CN, CF₃, Cl, Br, NRR, NO₂, unsubstituted phenyl and R is H or CH₃ .

19. The composition according to claim 17 wherein R^{A} and R^{B} are substituted phenyl groups, R¹, R² and R³ are independently selected from H, Phenyl, CN, CF₃, Cl, Br, OR, NRR, NO₂ and CH₃, R⁴, R⁵,R⁶, R⁹, R¹⁰, R¹¹ and R¹² are each H, R⁷ is phenyl or substituted phenyl, R⁸ is selected from H, CN, CF₃, Cl, Br, OR, NRR, NO₂, CH₃ and COOR and R is H or C₁-C₃ alkyl.

20. The composition according to claim 19 wherein when one of R¹, R² and R³ is other than H, the other of R¹, R² or R³ are H and R is H or CH₃.

21. The composition according to claim 20 wherein R¹ is NH₂, CN or Br, R⁷ is phenyl and R⁸ is COOR.

22. The composition according to claim 21 wherein R² and R³ are both H.

23. The composition according to 22 wherein R¹ is NH₂ and R is CH₃.

24. The composition according to claim 19 wherein R⁷ is phenyl, R⁸ is COOR and R is a C₁ to C₃ alkyl.

25. The composition according to claim 24 wherein R¹ and R² are H, R³ is H, CN, phenyl, CH₃, OCH₃, Br or Cl and R is CH₃.

26. The composition according to claim 25 wherein R³ is phenyl, Cl or CH₃.

27. The composition according to claim 26 wherein R³ is phenyl.

28. The composition according to claim 19 wherein R¹ and R³ are H, R² is CN, NO₂, CH₃, Cl, Br or CF₃, R⁷ is phenyl, and R is C₁-C₃ alkyl.

29. The composition according to claim 28 wherein R² is CH₃, Cl or Br and R is CH₃.

30. The composition according to claim 19 wherein R¹ and R² are H, R³ is CN, NO₂, CH₃, Cl, Phenyl, Br or CF₃, R⁷ is phenyl and R⁸ is H.

31. The composition according to claim 30 wherein R³ is CN or phenyl.

32. The composition according to claim 31 wherein R³ is phenyl.

33. A method of treating an infection in a patient caused by an agent selected from the group consisting of Trypanosoma cruzi, Mycobacterium spp., Leishmania spp., Cryptococcus spp., Aspergillus spp., Histoplasma spp., Candida spp., Pneumocystis carnii, Trichophyton spp., Microsporum spp. Malassezia spp., Rhizopus spp., Pseudallescheria spp., Blastomyces dermatitidis and Coccidiodes spp. comprising administering to said patient in need thereof an effective amount of a compound according to formula I: 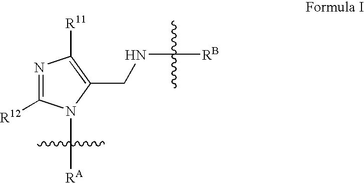
Where R^{A} is a C₁-C₁₀ substituted or unsubstituted linear, branch-chained or cyclic alkyl or alkenyl group or a phenyl group according to the formula: 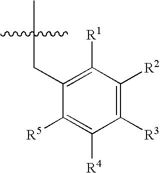
R^{B} is a C₁-C₁₀ substituted or unsubstituted linear, branch-chained or cyclic alkyl or alkenyl group or a phenyl group of the formula: 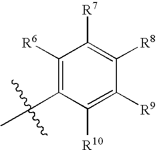
R¹, R², R³, R⁴, R⁵, R⁶, R⁷, R⁸, R⁹ and R¹⁰ are each independently selected from H, C₁-C₁₀ (preferably a C₁-C₄) alkyl or alkenyl group, CF₃, F, Cl, Br, I, CN, NO₂, NH₂, NHR, NRR, COR, OR, CO₂R, or COSR, where R is H or a C₁-C₁₀ (preferably a C₁-C₄) alkyl or alkenyl group, an unsubstituted or substituted aryl or heterocycle group,
or a 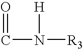
group, where R₃ is H, a C₁-C₁₀ (preferably a C₁-C₄) alkyl, alkenyl, ether or a thioether group; and
R¹¹ and R¹² are independently selected from H
or a C₁-C₃ alkyl or alkenyl group,
or a pharmaceutically acceptable salt thereof,
optionally in combination with a pharmaceutically acceptable additive carrier or excipient.

34. The method according to claim 33 wherein R^{A} and R^{B} are substituted phenyl groups, R¹, R², R³, R⁴, R⁵, R⁶, R⁷, R⁸, R⁹ and R¹⁰ are independently H or a C₁-C₃ alkyl group, CO₂R, OR, CN, CF₃, Cl, Br, NRR, NO₂, unsubstituted phenyl and R is H or CH₃.

35. The method according to claim 33 wherein R^{A} and R^{B} are substituted phenyl groups, R¹, R² and R³ are independently selected from H, Phenyl, CN, CF₃, Cl, Br, OR, NRR, NO₂ and CH₃, R⁴, R⁵, R⁶, R⁹, R¹⁰, R¹¹ and R¹² are each H, R⁷ is phenyl or substituted phenyl, R⁸ is selected from H, CN, CF₃, Cl, Br, OR, NRR, NO₂, CH₃ and COOR and R is H or C₁-C₃ alkyl.

36. The method according to claim 35 wherein when one of R¹, R² and R³ is other than H, the other of R¹, R² or R³ are H and R is H or CH₃.

37. The method according to claim 36 wherein R¹ is NH₂, CN or Br, R⁷ is phenyl and R⁸ is COOR.

38. The method according to claim 37 wherein R² and R³ are both H.

39. The method according to 38 wherein R¹ is NH₂ and R is CH₃.

40. The method according to claim 35 wherein R⁷ is phenyl, R⁸ is COOR and R is a C₁ to C₃ alkyl.

41. The method according to claim 40 wherein R¹ and R² are H, R³ is H, CN, phenyl, CH₃, OCH₃, Br or Cl and R is CH₃.

42. The method according to claim 41 wherein R³ is phenyl, Cl or CH₃.

43. The method according to claim 42 wherein R³ is phenyl.

44. The method according to claim 35 wherein R¹ and R³ are H, R² is CN, NO₂, CH₃, Cl, Br or CF₃, R⁷ is phenyl, and R is C₁-C₃ alkyl.

45. The method according to claim 43 wherein R² is CH₃, Cl or Br and R is CH₃.

46. The method according to claim 35 wherein R¹ and R² are H, R³ is CN, NO₂, CH₃, Cl, Phenyl, Br or CF₃ R⁷ is phenyl and R⁸ is H.

47. The method according to claim 46 wherein R³ is CN or phenyl.

48. The method according to claim 47 wherein R³ is phenyl.

49. The method according to claim 33 wherein said agent is Trypanosoma cruzi.

50. The method according to claim 35 wherein said agent is Trypanosoma cruzi.

51. The method according to claim 37 wherein said agent is Trypanosoma cruzi.

52. The method according to claim 43 wherein said agent is Trypanosoma cruzi.

53. The method according to claim 46 wherein said agent is Trypanosoma cruzi.

54. The method according to claim 33 wherein said agent is Candida albicans.

55. The method according to claim 54 wherein R¹, R², R⁴, R⁵, R⁶, R⁸, R⁹, R¹⁰ R¹¹ and R¹² are H, R⁷ is phenyl and R³ is CH₃.

56. A method of reducing the likelihood that a patient will contract an infection caused by an agent selected from the group consisting of Trypanosoma cruzi, Mycobacterium spp., Leishmania spp., Cryptococcus spp., Aspergillus spp., Histoplasma spp., Candida spp., Pneumocystis carinii, Trichophyton spp., Microsporum spp., Malassezia spp., Rhizopus spp., Pseudallescheria spp., Blastomyces dermatitidis and Coccidiodes spp., said method comprising administering to said patient in need thereof an effective amount of a compound according to formula I: 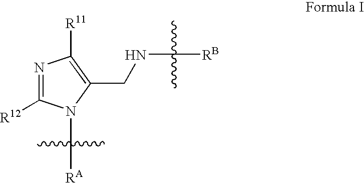
Where R^{A} is a C₁-C₁₀ substituted or unsubstituted linear, branch-chained or cyclic alkyl or alkenyl group or a phenyl group according to the formula: 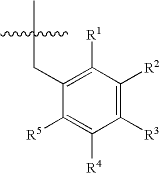
R^{B} is a C₁-C₁₀ substituted or unsubstituted linear, branch-chained or cyclic alkyl or alkenyl group or a phenyl group of the formula: 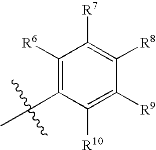
R¹, R², R³, R⁴, R⁵, R⁶, R⁷, R⁸, R⁹ and R¹⁰ are each independently selected from H, C₁-C₁₀ (preferably a C₁-C₄) alkyl or alkenyl group, CF₃, F, Cl, Br, I, CN, NO₂, NH₂, NHR, NRR, COR, OR, CO₂R, or COSR, where R is H or a C₁-C₁₀ (preferably a C₁-C₄) alkyl or alkenyl group, an unsubstituted or substituted aryl or heterocycle group,
or a 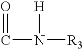
group, where R₃ is H, a C₁-C₁₀ (preferably a C₁-C₄) alkyl, alkenyl, ether or a thioether group; and
R¹¹ and R¹² are independently selected from H or a C₁-C₃ alkyl or alkenyl group,
or a pharmaceutically acceptable salt thereof,
optionally in combination with a pharmaceutically acceptable additive carrier or excipient.
